(19) United States
(12) Patent Application Publication
(10) Pub. No.: US 2024/0110174 A1
(43) Pub. Date: Apr. 4, 2024

(54) COMPOSITIONS, SYSTEMS, AND METHODS FOR TREATING CANCER USING ALTERNATING ELECTRIC FIELDS AND DENDRITIC CELLS

(71) Applicant: Novocure GmbH, Root D4 (CH)

(72) Inventors: Tali VOLOSHIN-SELA, Haifa (IL); Yiftah BARSHESHET, Haifa (IL); Gadi COHEN, Haifa (IL); IIan VOLOVITZ, Tel Aviv (IL); Gilad LEHMANN, Netanya (IL)

(73) Assignee: Novocure GmbH, Root D4 (CH)

(21) Appl. No.: 18/477,010

(22) Filed: Sep. 28, 2023

Related U.S. Application Data
(60) Provisional application No. 63/378,004, filed on Sep. 30, 2022.
(60) Provisional application No. 63/486,007, filed on Feb. 20, 2023.

(51) Int. Cl. C12N 13/00; A61K 39/00; C12N 5/0784

(52) U.S. Cl. CPC C12N 13/00; A61K 39/0011; C12N 5/0639; A61K 2039/5154; C12N 2501/052; C12N 2502/30; C12N 2529/00

(57) ABSTRACT

Compositions, systems, and methods for activating dendritic cells are disclosed. Also disclosed are compositions, systems, and methods for reducing viability of cancer cells and treating cancer, as well as preventing an increase in volume of a tumor present in a body of a living subject, along with methods of treating other diseases and infections. The systems and methods involve application of an alternating electric field to dendritic cell(s) in vivo or ex vivo and/or to a subject or cancer cells isolated therefrom. The systems and methods may further include administration of activated dendritic cells to a subject. The compositions comprise populations of isolated dendritic cells activated by exposure to an alternating electric field.

### CROSS-REFERENCE TO RELATED APPLICATIONS/INCORPORATION BY REFERENCE STATEMENT

The subject application claims benefit under 35 USC § 119(e) of U.S. Provisional Application No. 63/378,004, filed Sep. 30, 2022; and U.S. Provisional Application No. 63/486,007, filed Feb. 20, 2023. The entire contents of the above-referenced patent applications are hereby expressly incorporated herein by reference.

### STATEMENT REGARDING FEDERALLY SPONSORED RESEARCH OR DEVELOPMENT

Not Applicable.

### BACKGROUND

Tumor Treating Fields (TTFields) are low intensity (e.g., 1-3 V/cm) alternating electric fields within the intermediate frequency range (such as, but not limited to, 100-500 kHz) that target solid tumors by disrupting mitosis. This non-invasive treatment targets solid tumors and is described, for example, in U.S. Pat. Nos. 7,016,725; 7,089,054; 7,333,852; 7,565,205; 8,244,345; 8,715,203; 8,764,675; 10,188,851; and 10,441,776. TTFields are typically delivered through two pairs of transducer arrays that generate perpendicular fields within the treated tumor; the electrode arrays that make up each of these pairs are positioned on opposite sides of the body part that is being treated. More specifically, for the OPTUNE® system, one pair of electrodes is located to the left and right (LR) of the tumor, and the other pair of electrodes is located anterior and posterior (AP) to the tumor. TTFields are approved for the treatment of glioblastoma multiforme (GBM), and may be delivered, for example, via the OPTUNE® system (Novocure Limited, St. Helier, Jersey), which includes transducer arrays placed on the patient's shaved head.

Each transducer array used for the delivery of TTFields in the OPTUNE® device comprises a set of ceramic disk electrodes, which are coupled to the patient's skin (such as, but not limited to, the patient's shaved head for treatment of GBM) through a layer of conductive medical gel. The purpose of the medical gel is to deform to match the body's contours and to provide good electrical contact between the arrays and the skin; as such, the gel interface bridges the skin and reduces interference. The device is intended to be continuously worn by the patient for 2-4 days before removal for hygienic care and re-shaving (if necessary), followed by reapplication with a new set of arrays. As such, the medical gel remains in substantially continuous contact with an area of the patient's skin for a period of 2-4 days at a time, and there is only a brief period of time in which the area of skin is uncovered and exposed to the environment before more medical gel is applied thereto.

Another cancer treatment modality involves cancer immunotherapy. The primary goal of cancer immunotherapy is to activate a preexisting, endogenous immune response in cancer patients. Certain possible targets of cancer immunotherapy include mutation-derived tumor specific antigens, or neoantigens, which are absent from normal cells and can be recognized by the immune system, thereby providing a specific target for antitumor therapy. Although significant advances have been made in the field, treatment efficacy still needs to be improved.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 graphically depicts a representative dendritic cell (DC) gating strategy utilized in accordance with the present disclosure. Peripheral blood mononuclear cells (PBMCs) from the control and from the 150 kHz TTFields treatment groups were stained with the DC panel provided in Table 2, read by flow cytometry, and analyzed by FlowJo. The gating strategy is the same for all samples as seen in the first two rows. The third and fourth row show all viable DCs in 3 separate samples for control and 3 separate samples for TTFields.

FIG. 2 graphically depicts viability of dendritic cells in the different experimental groups. Mean viability is presented with standard errors of mean (SEMS) for each DC subtype based on 8 experiments, with 15 technical repeats per group in total.

FIG. 3 graphically depicts maturation of DCs following TTFields treatment in accordance with the present disclosure. Live DC of three subtypes (cDC1, cDC2 and pDC) were gated for two maturation markers, CD80 and CD83. From bottom to top of each 100% data bar, the individual portions of each data bar are as follows: (i) CD83+CD80+; (ii) CD83+; (iii) CD80+; and (iv) CD83−CD80−. The bars depict percent of cells that are either single positive (for one activation marker but not the other, exhibited as the second and third portions of each bar for the CD83+ and CD80+ cells, respectively) or double positive (CD83+CD80+, exhibited as the bottom portion of each data bar), which represents fully mature DC. Results represent mean of 8 experiments (15 technical repeats per each group). SEM was added to the double positive group.

FIG. 4 graphically depicts the fraction of double positive (CD80+, CD83+) cDC1 in the control (presented as the left data bar for each experiment) and 150 kHz TTFields-groups (presented as the right data bar for each experiment) across 8 experiments. Shown are either individual values or means of 2-3 repeats within a specific experiment. The mean of double activation in the 150 kHz group was higher than the control; however, the extent of differences across the experiments varies.

FIG. 5 graphically depicts the mean of cDC2 double positive (CD80+ and CD83+) in the control (presented as the left data bar for each experiment) and in the 150 kHz TTFields-treated group (presented as the right data bar for each experiment) across the 8 experiments performed. Shown are either individual values, or means of 2-3 repeats within a specific experiment.

FIG. 6 graphically depicts the mean of pDC double positive (CD80+ and CD83+) in the control (presented as the left data bar for each experiment) and in the 150 kHz TTFields-treated group (presented as the right data bar for each experiment) across the 8 experiments performed. Shown are either individual values or means of 2-3 repeats within a specific experiment. pDC double positive fractions were higher in the 150 kHz groups than the control samples (P=0.018).

### DETAILED DESCRIPTION

Before explaining at least one embodiment of the inventive concept(s) in detail by way of exemplary language and results, it is to be understood that the inventive concept(s) is not limited in its application to the details of construction and the arrangement of the components set forth in the following description. The inventive concept(s) is capable of other embodiments or of being practiced or carried out in various ways. As such, the language used herein is intended to be given the broadest possible scope and meaning; and the embodiments are meant to be exemplary—not exhaustive. Also, it is to be understood that the phraseology and terminology employed herein is for the purpose of description and should not be regarded as limiting.

Unless otherwise defined herein, scientific and technical terms used in connection with the presently disclosed inventive concept(s) shall have the meanings that are commonly understood by those of ordinary skill in the art. Further, unless otherwise required by context, singular terms shall include pluralities and plural terms shall include the singular. The foregoing techniques and procedures are generally performed according to conventional methods well known in the art and as described in various general and more specific references that are cited and discussed throughout the present specification. The nomenclatures utilized in connection with, and the laboratory procedures and techniques of, analytical chemistry, synthetic organic chemistry, and medicinal and pharmaceutical chemistry described herein are those well-known and commonly used in the art. Standard techniques are used for chemical syntheses and chemical analyses.

All patents, published patent applications, and non-patent publications mentioned in the specification are indicative of the level of skill of those skilled in the art to which this presently disclosed inventive concept(s) pertains. All patents, published patent applications, and non-patent publications referenced in any portion of this application are herein expressly incorporated by reference in their entirety to the same extent as if each individual patent or publication was specifically and individually indicated to be incorporated by reference.

All of the compositions, assemblies, systems, kits, and/or methods disclosed herein can be made and executed without undue experimentation in light of the present disclosure. While the compositions, assemblies, systems, kits, and methods of the inventive concept(s) have been described in terms of particular embodiments, it will be apparent to those of skill in the art that variations may be applied to the compositions and/or methods and in the steps or in the sequence of steps of the methods described herein without departing from the concept, spirit, and scope of the inventive concept(s). All such similar substitutions and modifications apparent to those skilled in the art are deemed to be within the spirit, scope, and concept of the inventive concept(s) as defined by the appended claims.

As utilized in accordance with the present disclosure, the following terms, unless otherwise indicated, shall be understood to have the following meanings:

The use of the term “a” or “an” when used in conjunction with the term “comprising” in the claims and/or the specification may mean “one,” but it is also consistent with the meaning of “one or more,” “at least one,” and “one or more than one.” As such, the terms “a,” “an,” and “the” include plural referents unless the context clearly indicates otherwise. Thus, for example, reference to “a compound” may refer to one or more compounds, two or more compounds, three or more compounds, four or more compounds, or greater numbers of compounds. The term “plurality” refers to “two or more.”

The use of the term “at least one” will be understood to include one as well as any quantity more than one, including but not limited to, 2, 3, 4, 5, 10, 15, 20, 30, 40, 50, 100, etc. The term “at least one” may extend up to 100 or 1000 or more, depending on the term to which it is attached; in addition, the quantities of 100/1000 are not to be considered limiting, as higher limits may also produce satisfactory results. In addition, the use of the term “at least one of X, Y, and Z” will be understood to include X alone, Y alone, and Z alone, as well as any combination of X, Y, and Z. The use of ordinal number terminology (i.e., “first,” “second,” “third,” “fourth,” etc.) is solely for the purpose of differentiating between two or more items and is not meant to imply any sequence or order or importance to one item over another or any order of addition, for example.

The use of the term “or” in the claims is used to mean an inclusive “and/or” unless explicitly indicated to refer to alternatives only or unless the alternatives are mutually exclusive. For example, a condition “A or B” is satisfied by any of the following: A is true (or present) and B is false (or not present), A is false (or not present) and B is true (or present), and both A and B are true (or present).

As used herein, any reference to “one embodiment,” “an embodiment,” “some embodiments,” “one example,” “for example,” or “an example” means that a particular element, feature, structure, or characteristic described in connection with the embodiment is included in at least one embodiment. The appearance of the phrase “in some embodiments” or “one example” in various places in the specification is not necessarily all referring to the same embodiment, for example. Further, all references to one or more embodiments or examples are to be construed as non-limiting to the claims.

Throughout this application, the term “about” is used to indicate that a value includes the inherent variation of error for a composition/apparatus/device, the method being employed to determine the value, or the variation that exists among the study subjects. For example, but not by way of limitation, when the term “about” is utilized, the designated value may vary by plus or minus twenty percent, or fifteen percent, or twelve percent, or eleven percent, or ten percent, or nine percent, or eight percent, or seven percent, or six percent, or five percent, or four percent, or three percent, or two percent, or one percent from the specified value, as such variations are appropriate to perform the disclosed methods and as understood by persons having ordinary skill in the art.

As used in this specification and claim(s), the words “comprising” (and any form of comprising, such as “comprise” and “comprises”), “having” (and any form of having, such as “have” and “has”), “including” (and any form of including, such as “includes” and “include”), or “containing” (and any form of containing, such as “contains” and “contain”) are inclusive or open-ended and do not exclude additional, unrecited elements or method steps.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, AAB, BBC, AAABCCCC, CBBAAA, CABABB, and so forth. The skilled artisan will understand that typically there is no limit on the number of items or terms in any combination, unless otherwise apparent from the context.

As used herein, the term “substantially” means that the subsequently described event or circumstance completely occurs or that the subsequently described event or circumstance occurs to a great extent or degree. For example, when associated with a particular event or circumstance, the term “substantially” means that the subsequently described event or circumstance occurs at least 80% of the time, or at least 85% of the time, or at least 90% of the time, or at least 95% of the time. For example, the term “substantially adjacent” may mean that two items are 100% adjacent to one another, or that the two items are within close proximity to one another but not 100% adjacent to one another, or that a portion of one of the two items is not 100% adjacent to the other item but is within close proximity to the other item.

The term “pharmaceutically acceptable” refers to compounds and compositions which are suitable for administration to humans and/or animals without undue adverse side effects such as (but not limited to) toxicity, irritation, and/or allergic response commensurate with a reasonable benefit/risk ratio.

The term “patient” or “subject” as used herein includes human and veterinary subjects. “Mammal” for purposes of treatment refers to any animal classified as a mammal, including (but not limited to) humans, domestic and farm animals, nonhuman primates, and any other animal that has mammary tissue.

The term “treatment” refers to both therapeutic treatment and prophylactic or preventative measures. Those in need of treatment include, but are not limited to, individuals already having a particular condition/disease/infection as well as individuals who are at risk of acquiring a particular condition/disease/infection (e.g., those needing prophylactic/preventative measures). The term “treating” refers to administering an agent/element/method to a patient for therapeutic and/or prophylactic/preventative purposes.

The term “therapeutic composition” or “pharmaceutical composition” as used herein refers to an agent that may be administered in vivo to bring about a therapeutic and/or prophylactic/preventative effect.

Administering a therapeutically effective amount or prophylactically effective amount is intended to provide a therapeutic benefit in the treatment, prevention, and/or management of a disease, condition, and/or infection. The specific amount that is therapeutically effective can be readily determined by the ordinary medical practitioner, and can vary depending on factors known in the art, such as (but not limited to) the type of condition/disease/infection, the patient's history and age, the stage of the condition/disease/infection, and the co-administration of other agents.

The term “effective amount” refers to an amount of a biologically active molecule or conjugate or derivative thereof, or an amount of a treatment protocol (i.e., an alternating electric field), sufficient to exhibit a detectable therapeutic effect without undue adverse side effects (such as (but not limited to) toxicity, irritation, and allergic response) commensurate with a reasonable benefit/risk ratio when used in the manner of the inventive concept(s). The therapeutic effect may include, for example but not by way of limitation, preventing, inhibiting, or reducing the occurrence of at least one condition, disease, and/or infection. The effective amount for a subject will depend upon the type of subject, the subject's size and health, the nature and severity of the condition/disease/infection to be treated, the method of administration, the duration of treatment, the nature of concurrent therapy (if any), the specific formulations employed, and the like. Thus, it is not possible to specify an exact effective amount in advance. However, the effective amount for a given situation can be determined by one of ordinary skill in the art using routine experimentation based on the information provided herein.

As used herein, the term “concurrent therapy” is used interchangeably with the terms “combination therapy” and “adjunct therapy,” and will be understood to mean that the patient in need of treatment is treated or given another drug for the condition/disease/infection in conjunction with the treatments of the present disclosure. This concurrent therapy can be sequential therapy, where the patient is treated first with one treatment protocol/pharmaceutical composition and then the other treatment protocol/pharmaceutical composition, or the two treatment protocols/pharmaceutical compositions are given simultaneously.

The terms “administration” and “administering,” as used herein, will be understood to include all routes of administration known in the art, including but not limited to, oral, topical, transdermal, parenteral, subcutaneous, intranasal, mucosal, intramuscular, intraperitoneal, intravitreal, and intravenous routes, and including both local and systemic applications. In addition, the compositions of the present disclosure (and/or the methods of administration of same) may be designed to provide delayed, controlled, or sustained release using formulation techniques which are well known in the art.

Turning now to the inventive concept(s), methods of maturing and activating dendritic cells, along with the use of such activated dendritic cells in the treatment of a subject, are disclosed herein. The methods involve application of an alternating electric field (such as, but not limited to, TTFields) to immature dendritic cells or precursors thereof to mature/activate the dendritic cells. The methods may further include a step of loading the dendritic cells with antigens from a particular source (such as, but not limited to, cancer antigens, viral antigens, bacterial antigens, fungal antigens, etc.). Then the activated, antigen-loaded dendritic cells can be administered to a subject for treatment of a condition, infection, or disease.

In a particular (but non-limiting) embodiment, the dendritic cells are loaded with antigens from cancer cells, including, but not limited to, cancer cells that have also been exposed to alternating electric fields (such as, but not limited to, TTFields), either in vivo or ex vivo. Administration of the activated, antigen-loaded dendritic cells to the cancer subject provides a synergistic result in the treatment of cancer.

The inventive concepts also include a combinatorial therapy for cancer that combines (i) production of alternating electric field—(such as, but not limited to, TTField—) treated cancer cells via application of alternating electric fields (such as, but not limited to, TTFields) to either a subject or ex vivo to cells isolated from the subject; (ii) use of these alternating electric field-treated cancer cells to activate dendritic cells (i.e., load the dendritic cells ex vivo with antigens from the alternating electric field-treated cancer cells); and (iii) administration to the subject of at least one composition that contains the activated, antigen-loaded dendritic cells. The combination of alternating electric fields with composition(s) containing dendritic cells activated by co-culture with alternating electric field-treated cancer cells provides a synergistic result in the treatment of cancer.

Certain non-limiting embodiments of the present disclosure are directed to a method of activating dendritic cells, that includes applying an alternating electric field to a composition comprising immature dendritic cells or precursors thereof in vitro for a period of time sufficient to produce activated dendritic cells. The method may further comprise the step of contacting the activated dendritic cells with a source of antigens to produce antigen-loaded dendritic cells.

Certain non-limiting embodiments of the present disclosure are directed to a method of preparing an immunogenic composition, wherein the method includes the steps of: applying an alternating electric field to a composition comprising immature dendritic cells or precursors thereof in vitro for a period of time sufficient to produce activated dendritic cells; contacting the activated dendritic cells with a source of antigens to produce antigen-loaded dendritic cells; and isolating the antigen-loaded dendritic cells to form the immunogenic composition. The dendritic cells may be pulsed with antigens (such as, but not limited to, bacterial, viral, fungal, tumor, and/or cancer antigens) or co-cultured with a source of antigens; for example (but not by way of limitation), the dendritic cells may be co-cultured with at least one cancer cell isolated from the subject to produce antigen-loaded dendritic cells.

Certain non-limiting embodiments of the present disclosure are directed to a method of preparing an immunogenic composition. The method includes the steps of: (1) applying an alternating electric field ex vivo to a composition comprising immature dendritic cells and/or dendritic cell precursors to produce mature dendritic cells; (2) co-culturing the mature dendritic cells with at least one cancer cell isolated from the subject to produce antigen-loaded dendritic cells; and (3) isolating the antigen-loaded dendritic cells from the co-culture of (2) and from the at least one cancer cell to form the immunogenic composition.

Certain non-limiting embodiments of the present disclosure are directed to a method of treating cancer in a subject. The method includes the steps of: (1) applying an alternating electric field ex vivo to a composition comprising immature dendritic cells and/or dendritic cell precursors to produce mature dendritic cells; (2) co-culturing the mature dendritic cells with at least one cancer cell isolated from the subject to produce antigen-loaded dendritic cells; (3) isolating the antigen-loaded dendritic cells from the co-culture of (2) and from the at least one cancer cell; and (4) administering the antigen-loaded dendritic cells to the subject.

Certain additional non-limiting embodiments of the present disclosure are directed to a method of reducing a volume of a tumor present in a body of a living subject, wherein the tumor includes a plurality of cancer cells. The method includes the steps of: (1) applying an alternating electric field ex vivo to a composition comprising immature dendritic cells and/or dendritic cell precursors to produce mature dendritic cells; (2) co-culturing the mature dendritic cells with at least one cancer cell isolated from the tumor of the subject to produce antigen-loaded dendritic cells; (3) isolating the antigen-loaded dendritic cells from the co-culture of (2) and from the at least one cancer cell; and (4) administering the antigen-loaded dendritic cells to the subject.

Certain additional non-limiting embodiments of the present disclosure are directed to a method of preventing an increase of volume of a tumor present in a body of a living subject, wherein the tumor includes a plurality of cancer cells. The method includes the steps of: (1) applying an alternating electric field ex vivo to a composition comprising immature dendritic cells and/or dendritic cell precursors to produce mature dendritic cells; (2) co-culturing the mature dendritic cells with at least one cancer cell isolated from the tumor of the subject to produce antigen-loaded dendritic cells; (3) isolating the antigen-loaded dendritic cells from the co-culture of (2) and from the at least one cancer cell; and (4) administering the antigen-loaded dendritic cells to the subject.

In certain particular (but non-limiting) embodiments of the present disclosure, the at least one cancer cell is also exposed to an alternating electric field. This exposure may occur during the co-culture step; that is, at least a portion of steps (1) and (2) of any of the methods disclosed herein above or otherwise contemplated herein can be performed simultaneously, whereby the alternating electric field is also applied to the at least one cancer cell during the co-culture. Alternatively (and/or in addition thereto), this exposure may occur prior to contact with the dendritic cells/precursors. For example (but not by way of limitation), an alternating electric field may be applied to a target region of the subject prior to isolation of the at least one cancer cell from the subject, and/or the cancer cell(s) isolated from the subject may be exposed to an alternating electric field ex vivo and prior to co-culture.

Any of the methods disclosed or otherwise contemplated herein may further include, in certain non-limiting embodiments, step (5) of applying the alternating electric field to the target region of the subject following administration of the activated, antigen-loaded dendritic cells.

Certain non-limiting embodiments of the present disclosure are directed to a method of preparing an immunogenic composition. The method includes the steps of: co-culturing dendritic cells with at least one cancer cell isolated from a subject to produce antigen-loaded dendritic cells, wherein the at least one cancer cell has been exposed to an alternating electric field in vivo or ex vivo prior to co-culture with the dendritic cells; and isolating a population of antigen-loaded dendritic cells to form the immunogenic composition.

Certain non-limiting embodiments of the present disclosure are directed to a method of preparing an immunogenic composition. The method includes the steps of: (1) applying an alternating electric field to a target region of the subject; (2) isolating cancer cells from the target region to which the alternating electric field has been applied; (3) co-culturing the isolated cancer cells with dendritic cells to produce activated, antigen-loaded dendritic cells; and (4) isolating the activated, antigen-loaded dendritic cells from the co-culture of (3) and from the cancer cells present therein to form the immunogenic composition.

Certain non-limiting embodiments of the present disclosure are directed to a method of treating cancer in a subject. The method includes the steps of: (1) applying an alternating electric field to a target region of the subject; (2) isolating cancer cells from the target region to which the alternating electric field has been applied; (3) co-culturing the isolated cancer cells with dendritic cells to produce activated, antigen-loaded dendritic cells; (4) isolating the activated, antigen-loaded dendritic cells from the co-culture of (3) and from the cancer cells present therein; and (5) administering the activated, antigen-loaded dendritic cells to the subject.

Certain additional non-limiting embodiments of the present disclosure are directed to a method of preparing an immunogenic composition. The method includes the steps of: (1) isolating at least one cancer cell from the subject (such as, but not limited to, from at least a portion of a tumor in the subject); (2) applying an alternating electric field ex vivo to the isolated at least one cancer cell; (3) co-culturing the isolated at least one cancer cell to which the alternating electric field has been applied with dendritic cells to produce activated, antigen-loaded dendritic cells; and (4) isolating the activated, antigen-loaded dendritic cells from the co-culture of (3) and from the at least one cancer cell present therein to form the immunogenic composition.

Certain additional non-limiting embodiments of the present disclosure are directed to a method of treating cancer in a subject. The method includes the steps of: (1) isolating at least one cancer cell from the subject (such as, but not limited to, from at least a portion of a tumor in the subject); (2) applying an alternating electric field ex vivo to the isolated at least one cancer cell; (3) co-culturing the isolated at least one cancer cell to which the alternating electric field has been applied with dendritic cells to produce activated, antigen-loaded dendritic cells; (4) isolating the activated, antigen-loaded dendritic cells from the co-culture of (3) and from the at least one cancer cell present therein; and (5) administering the activated, antigen-loaded dendritic cells to the subject.

Certain additional non-limiting embodiments of the present disclosure are directed to a method of reducing a volume of a tumor present in a body of a living subject, wherein the tumor includes a plurality of cancer cells. The method includes the steps of: (1) applying an alternating electric field to a target region of the subject, wherein the target region includes the tumor; (2) isolating cancer cells from the target region to which the alternating electric field has been applied; (3) co-culturing the isolated cancer cells with dendritic cells to produce activated, antigen-loaded dendritic cells; (4) isolating the activated, antigen-loaded dendritic cells from the co-culture of (3) and from the cancer cells present therein; and (5) administering the activated, antigen-loaded dendritic cells to the subject.

Certain additional non-limiting embodiments of the present disclosure are directed to a method of reducing a volume of a tumor present in a body of a living subject, wherein the tumor includes a plurality of cancer cells. The method includes the steps of: (1) isolating at least one cancer cell from the subject (such as, but not limited to, from at least a portion of a tumor in the subject); (2) applying an alternating electric field ex vivo to the isolated at least one cancer cell; (3) co-culturing the isolated at least one cancer cell to which the alternating electric field has been applied with dendritic cells to produce activated, antigen-loaded dendritic cells; (4) isolating the activated, antigen-loaded dendritic cells from the co-culture of (3) and from the at least one cancer cell present therein; and (5) administering the activated, antigen-loaded dendritic cells to the subject.

Certain additional non-limiting embodiments of the present disclosure are directed to a method of preventing an increase of volume of a tumor present in a body of a living subject, wherein the tumor includes a plurality of cancer cells. The method includes the steps of: (1) applying an alternating electric field to a target region of the subject, wherein the target region includes the tumor; (2) isolating cancer cells from the target region to which the alternating electric field has been applied; (3) co-culturing the isolated cancer cells with dendritic cells to produce activated, antigen-loaded dendritic cells; (4) isolating the activated, antigen-loaded dendritic cells from the co-culture of (3) and from the cancer cells present therein; and (5) administering the activated, antigen-loaded dendritic cells to the subject.

Certain additional non-limiting embodiments of the present disclosure are directed to a method of preventing an increase of volume of a tumor present in a body of a living subject, wherein the tumor includes a plurality of cancer cells. The method includes the steps of: (1) isolating at least one cancer cell from the subject (such as, but not limited to, from at least a portion of a tumor in the subject); (2) applying an alternating electric field ex vivo to the isolated at least one cancer cell; (3) co-culturing the isolated at least one cancer cell to which the alternating electric field has been applied with dendritic cells to produce activated, antigen-loaded dendritic cells; (4) isolating the activated, antigen-loaded dendritic cells from the co-culture of (3) and from the at least one cancer cell present therein; and (5) administering the activated, antigen-loaded dendritic cells to the subject.

Any of the above methods disclosed or otherwise contemplated herein may further include, in certain non-limiting embodiments, step (6) of applying an alternating electric field to the target region of the subject following administration of the activated, antigen-loaded dendritic cells.

Following alternating electric field-treatment, the treated cancer cells that are subsequently utilized in the co-culture step may have any viability state. That is, regardless of whether the cells are viable, apoptotic, and/or non-viable, the treated cancer cells used in the co-culture step will be capable of triggering maturation of dendritic cells in the co-culture step.

The dendritic cells/precursors thereof utilized may be obtained from the subject or from another source, such as (but not limited to) an HLA-matched donor. For example (but not by way of limitation), the method may further comprise the step of isolating dendritic cells or precursors thereof from the subject. When the method includes applying an alternating electric field directly to the subject, the dendritic cells or precursors thereof may be isolated from the subject before or after application of the alternating electric field.

In another particular (but non-limiting) embodiment, the dendritic cells or precursors thereof are isolated from an HLA-matched donor. For example (but not by way of limitation), when a patient is treated with immunosuppressants or similar technologies (e.g., a CRISPR technique to reduce allogeneic reaction of non-matched donors), an HLA-matched donor can be used for isolation of the dendritic cells.

In a particular (but non-limiting) embodiment, the method further includes the steps of isolating immature monocytes (or other dendritic cell precursors) from the blood stream of the subject or a donor (such as, but not limited to, an HLA-matched donor); and generating immature dendritic cells from the immature monocytes/dendritic cell precursors.

In a particular (but non-limiting) embodiment, the composition containing dendritic cells/precursors thereof comprises peripheral blood mononuclear cells (PBMCs), isolated either from the subject or an HLA-matched donor.

The co-culturing step may be performed under any conditions that allow for loading of the dendritic cells with antigens from the cancer cells. In certain particular (but non-limiting) embodiments, the co-culturing step is performed in the presence of at least one composition selected from the group consisting of a cytokine, an interferon, granulocyte-macrophage colony-stimulating factor (GM-CSF), CD40 ligand (CD40L), a Toll-like receptor (TLR) agonist, and the like, as well as any combinations thereof. In addition, the co-culturing step may be performed in the presence or absence of application of an alternating electric field thereto.

The activated, antigen-loaded dendritic cells may be isolated from the co-culture and the cancer cells present therein using any methods known in the art or otherwise contemplated herein. The isolation of the antigen-loaded dendritic cells may be accomplished in a single step or in multiple steps. For example (but not by way of limitation), the cells may first be isolated from the co-culture by general cell isolation methods, and then a second, specific isolation step (such as, but not limited to, a percol/ficoll gradient, flow cytometry sorting, bead sorting, etc.) may be utilized to ensure that all cancer cells are removed and only antigen-loaded dendritic cells remain.

It should be noted that the isolated dendritic cells that are administered to the subject may also contain non-loaded cells in addition to the antigen-loaded dendritic cells. As such, the composition administered to the subject may also be referred to herein as “co-cultured dendritic cells” or “antigen-experienced dendritic cells.”

The compositions and methods of the present disclosure may be utilized with any types of cancer cells and/or to treat any types of cancer cells/cancers/tumors, such as (but not limited to) those cancers that respond to alternating electric field and/or activated dendritic cell treatment. Non-limiting examples of cancer cells/cancers/tumors that can be utilized in accordance with the present disclosure include hepatocellular carcinomas/carcinoma cells, glioblastomas/glioblastoma cells, pleural mesotheliomas/mesothelioma cells, differentiated thyroid cancers/cancer cells, advanced renal cell carcinomas/carcinoma cells, ovarian cancers/cancer cells, cervical cancers/cancer cells, breast cancers/cancer cells, pancreatic cancers/cancer cells, lung cancers/cancer cells (such as, but not limited to, non-small cell lung cancers/cancer cells), and the like, as well as any combination thereof.

In a particular (but non-limiting) embodiment, the cancer cell(s) utilized in accordance with the present disclosure may be taken from at least a portion of a tumor.

In a particular (but non-limiting) embodiment, the cancer may be a solid tumor.

Any type of conductive or non-conductive electrode(s) and/or transducer array(s) that can be utilized for generating an alternating electric field that are known in the art or otherwise contemplated herein may be utilized for generation of the alternating electric field in accordance with the methods of the present disclosure. Non-limiting examples of electrodes and transducer arrays that can be utilized for generating an alternating electric field in accordance with the present disclosure include those that function as part of an alternating electric field-generating system (i.e., TTFields system) as described, for example but not by way of limitation, in U.S. Pat. Nos. 7,016,725; 7,089,054; 7,333,852; 7,565,205; 8,244,345; 8,715,203; 8,764,675; 10,188,851; and 10,441,776; and in US Patent Application Nos. US 2018/0160933; US 2019/0117956; US 2019/0307781; and US 2019/0308016.

The alternating electric field may be generated at any frequency in accordance with the present disclosure. For example (but not by way of limitation), the alternating electric field may have a frequency of about 50 kHz, about 60 kHz, about 70 kHz, about 75 kHz, about 80 kHz, about 90 kHz, about 100 kHz, about 105 kHz, about 110 kHz, about 115 kHz, about 120 kHz, about 125 kHz, about 130 kHz, about 135 kHz, about 140 kHz, about 145 kHz, about 150 kHz, about 155 kHz, about 160 kHz, about 165 kHz, about 170 kHz, about 175 kHz, about 180 kHz, about 185 kHz, about 190 kHz, about 195 kHz, about 200 kHz, about 225 kHz, about 250 kHz, about 275 kHz, about 300 kHz, about 325 kHz, about 350 kHz, about 375 kHz, about 400 kHz, about 425 kHz, about 450 kHz, about 475 kHz, about 500 kHz, about 550 kHz, about 600 kHz, about 650 kHz, about 700 kHz, about 750 kHz, about 800 kHz, about 850 kHz, about 900 kHz, about 950 kHz, about 1 MHz, and the like, as well as a range formed from any of the above values (e.g., a range of from about 50 kHz to about 1 MHz, a range of from about 100 kHz to about 500 kHz, a range of from about 150 kHz to about 300 kHz, a range of from about 50 kHz to about 190 kHz, a range of from about 50 kHz to about 180 kHz, a range of from about 50 kHz to about 175 kHz, a range of from about 50 kHz to about 160 kHz, a range of from about 50 kHz to about 150 kHz, a range of from about 250 kHz to about 350 kHz, a range of from about 350 kHz to about 500 kHz, a range of from about 250 kHz to about 500 kHz, etc.), and a range that combines two integers that fall between two of the above-referenced values (e.g., a range of from about 122 kHz to about 313 kHz, a range of from about 78 kHz to about 298 kHz, etc.).

In certain particular (but non-limiting) embodiments, the alternating electric field may be imposed at two or more different frequencies. When two or more frequencies are present, each frequency is selected from any of the above-referenced values, or a range formed from any of the above-referenced values, or a range that combines two integers that fall between two of the above-referenced values.

The alternating electric field may have any field strength in the subject/cancer cells, so long as the alternating electric field is capable of functioning in accordance with the present disclosure. For example (but not by way of limitation), the alternating electric field may have a field strength of at least about 1 V/cm, about 1.5 V/cm, about 2 V/cm, about 2.1 V/cm, about 2.2 V/cm, about 2.3 V/cm, about 2.4 V/cm, about 2.5 V/cm, about 2.6 V/cm, about 2.7 V/cm, about 2.8 V/cm, about 2.9 V/cm, about 3 V/cm, about 3.5 V/cm, about 4 V/cm, about 4.5 V/cm, about 5 V/cm, about 5.5 V/cm, about 6 V/cm, about 6.5 V/cm, about 7 V/cm, about 7.5 V/cm, about 8 V/cm, about 9 V/cm, about 9.5 V/cm, about 10 V/cm, about 10.5 V/cm, about 11 V/cm, about 11.5 V/cm, about 12 V/cm, about 12.5 V/cm, about 13 V/cm, about 13.5 V/cm, about 14 V/cm, about 14.5 V/cm, about 15 V/cm, about 15.5 V/cm, about 16 V/cm, about 16.5 V/cm, about 17 V/cm, about 17.5 V/cm, about 18 V/cm, about 18.5 V/cm, about 19 V/cm, about 19.5 V/cm, about 20 V/cm, and the like, as well as a range formed from any of the above values (e.g., a range of from about 1 V/cm to about 20 V/cm, a range of from about 1 V/cm to about 10 V/cm, a range of from about 1 V/cm to about 4 V/cm, etc.), and a range that combines two integers that fall between two of the above-referenced values (e.g., a range of from about 1.1 V/cm to about 18.6 V/cm, a range of from about 1.2 V/cm to about 9.8 V/cm, a range of from about 1.3 V/cm to about 4.7 V/cm, etc.).

The alternating electric field may be applied in a single direction between a pair of arrays or may be alternating in two (or more) directions between two (or more) pairs of arrays (e.g., front-back and left-right). For example, certain TTFields devices (such as, but not limited to, the OPTUNE® system (Novocure Limited, St. Helier, Jersey)) operate in two directions in order to increase the chances that a dividing cell will be aligned with the electric field such that the electric field can have the desired anti-mitotic effect. However, it will be understood that the scope of the present disclosure also includes the application of the alternating electric field in a single direction, in order to achieve the immunogenic response described herein.

The alternating electric field may be applied to the subject, the dendritic cells, and/or the cancer cells (or a co-culture containing both dendritic and cancer cells) for any period of time disclosed or otherwise contemplated herein. For example, but not by way of limitation, in certain non-limiting embodiments, the alternating electric field is applied for a period of time sufficient to cause/aid in maturation of the dendritic cells and/or cause/aid in presentation of certain antigens from the co-cultured cancer cells by the dendritic cells. For example, but not by way of limitation, the alternating electric field may be applied for at least about 1 hour, about 2 hours, about 3 hours, about 4 hours, about 5 hours, about 6 hours, about 7 hours, about 8 hours, about 9 hours, about 10 hours, about 11 hours, about 12 hours, about 15 hours, about 18 hours, about 21 hours, about 24 hours, about 27 hours, about 30 hours, about 33 hours, about 36 hours, about 39 hours, about 42 hours, about 45 hours, about 48 hours, about 51 hours, about 54 hours, about 57 hours, about 60 hours, about 63 hours, about 66 hours, about 69 hours, about 72 hours, about 75 hours, about 78 hours, about 81 hours, about 84 hours, about 87 hours, about 90 hours, about 93 hours, about 96 hours, about 1 day, about 2 days, about 3 days, about 4 days, about 5 days, about 6 days, about 7 days, and the like, as well as a range formed from any of the above values (e.g., a range of from about 1 minute to about 12 hours, a range of from about 1 minute to about 1 hour, a range of from about 1 hour to about 7 days, a range of from about 24 hours to about 72 hours, etc.), and a range that combines two integers that fall between two of the above-referenced values (e.g., a range of from about 14 hours to about 68 hours, etc.).

In a particular (but non-limiting) embodiment, the period of time that the alternating electric field is applied is at least about 24 hours.

In addition, when the alternating electric field is applied to a subject, the period of time that the alternating electric field is applied may be a continuous period of time or a cumulative period of time. That is, the period of time that the alternating electric field is applied may include a single session (i.e., continuous application) as well as multiple sessions with minor breaks in between sessions (i.e., consecutive application for a cumulative period). For example, a subject is allowed to take breaks during treatment with an alternating electric field device and is only expected to have the device positioned on the body and operational for at least about 50%, at least about 60%, at least about 70%, or at least about 80% of the total treatment period (e.g., over a course of one day, one week, two weeks, one month, two months, three months, four months, five months, etc.).

The antigen-loaded dendritic cells (referred to hereinafter as “activated dendritic cells”) may be disposed and administered in any formulation known in the art or otherwise contemplated herein that will allow the activated dendritic cells to have a deleterious effect on the cancer present in the subject. For example, but not by way of limitation, the activated dendritic cells may be administered in the form of a pharmaceutical composition that comprises the activated dendritic cells in combination with at least one pharmaceutically-acceptable carrier. Non-limiting examples of suitable pharmaceutically acceptable carriers that may be utilized in accordance with the present disclosure include water; saline; dextrose solutions; fructose or mannitol; calcium carbonate; cellulose; ethanol; oils of animal, vegetative, or synthetic origin; carbohydrates, such as glucose, sucrose, or dextrans; antioxidants, such as ascorbic acid or glutathione; chelating agents; low molecular weight proteins; detergents; liposomal carriers; conductive and non-conductive nanoparticles; buffered solutions, such as sodium chloride, saline, phosphate-buffered saline, and/or other substances which are physiologically acceptable and/or safe for use; diluents; excipients such as polyethylene glycol (PEG); or any combination thereof. Suitable pharmaceutically acceptable carriers for pharmaceutical formulations are described, for example, in Remington: The Science and Practice of Pharmacy, 23rd ed (2020).

In certain non-limiting embodiments, the pharmaceutical composition containing the activated dendritic cells may further be formulated as an immunogenic composition. The immunogenic composition may contain the same components as the pharmaceutical composition described above (i.e., the activated dendritic cells plus the pharmaceutically-acceptable carrier). In certain particular (but non-limiting) embodiments, the immunogenic composition may further include at least one additional agent. Non-limiting examples of agents that may be included as part of the immunogenic composition include an adjuvant, a cytokine, an interferon, a TLR agonist, a STING (stimulator of interferon genes) agonist, GM-CSF, CD40L, Fms related tyrosine kinase 3 ligand (FLT3L), a C type Lectin Receptor (CLR), an anti-LAG3 agent (such as, but not limited to, OPDUALAG™ and/or Relatimab (Bristol-Myers Squibb, New York, NY)), other active agents, and the like, as well as any combinations thereof.

In addition, any of the activated dendritic cell-containing compositions of the present disclosure may contain other agents that allow for administration of the compositions via a particular administration route. For example, but not by way of limitation, the compositions may be formulated for administration by oral, topical, transdermal, parenteral, subcutaneous, intranasal, mucosal, intramuscular, intraperitoneal, intravitreal, and/or intravenous routes. Based on the route of administration, the compositions may also contain one or more additional components in addition to the active agent (i.e., immunogenic composition and/or additional therapeutic agent). Examples of additional secondary compounds that may be present include, but are not limited to, fillers, gels, adhesives, salts, buffers, preservatives, stabilizers, solubilizers, wetting agents, emulsifying agents, dispersing agents, and other materials well known in the art.

In a particular (but non-limiting) embodiment, the composition containing the activated dendritic cells is administered intradermally, subcutaneously, intravenously, and/or intranodally to the subject.

In certain non-limiting embodiments, the method may further include one or more additional steps of applying the alternating electric field to the target region of the subject: (1) following isolation of the dendritic cells and/or precursors thereof; (2) following isolation of the cancer cells (and/or resection of the tumor); and/or (3) prior to or following administration of the activated dendritic cell-containing composition. When the additional alternating electric field application step(s) is present, the alternating electric field may be applied simultaneously or wholly or partially sequentially with the administration of the activated dendritic cell-containing composition. In certain particular (but non-limiting) embodiments, the alternating electric field may be applied after the activated dendritic cell-containing composition is administered. In other particular (but non-limiting) embodiments, the alternating electric field may be applied at the same time or after administration of the activated dendritic cell-containing composition. In another particular (but non-limiting) embodiment, the activated dendritic cell-containing composition may be administered during application of the alternating electric field (i.e., before the period of time that the alternating electric field is applied has elapsed).

For example (but not by way of limitation), the activated dendritic cell-containing composition may be administered before the additional application of the alternating electric field has commenced by a period of at least about 1 minute, about 5 minutes, about 10 minutes, about 15 minutes, about 30 minutes, about 45 minutes, about 1 hour, about 1.5 hours, about 2 hours, about 3 hours, about 4 hours, about 5 hours, about 6 hours, about 7 hours, about 8 hours, about 9 hours, about 10 hours, about 12 hours, about 15 hours, about 18 hours, about 21 hours, about 24 hours, about 27 hours, about 30 hours, about 33 hours, about 36 hours, about 39 hours, about 42 hours, about 45 hours, about 48 hours, about 51 hours, about 54 hours, about 57 hours, about 60 hours, about 63 hours, about 66 hours, about 69 hours, about 72 hours, about 75 hours, about 78 hours, about 81 hours, about 84 hours, about 87 hours, about 90 hours, about 93 hours, about 96 hours, and the like, as well as a range formed from any of the above values (e.g., a range of from about 1 minute to about 24 hours, etc.), and a range that combines two integers that fall between two of the above-referenced values (e.g., a range of from about 14 minutes to about 94 hours, etc.).

In other non-limiting examples, the activated dendritic cell-containing composition may be administered after the additional application of the alternating electric field has commenced by a period of at least about 1 minute, about 5 minutes, about 10 minutes, about 15 minutes, about 30 minutes, about 45 minutes, about 1 hour, about 1.5 hours, about 2 hours, about 3 hours, about 4 hours, about 5 hours, about 6 hours, about 7 hours, about 8 hours, about 9 hours, about 10 hours, about 12 hours, about 15 hours, about 18 hours, about 21 hours, about 24 hours, about 27 hours, about 30 hours, about 33 hours, about 36 hours, about 39 hours, about 42 hours, about 45 hours, about 48 hours, about 51 hours, about 54 hours, about 57 hours, about 60 hours, about 63 hours, about 66 hours, about 69 hours, about 72 hours, about 75 hours, about 78 hours, about 81 hours, about 84 hours, about 87 hours, about 90 hours, about 93 hours, about 96 hours, and the like, as well as a range formed from any of the above values (e.g., a range of from about 1 minute to about 24 hours, etc.), and a range that combines two integers that fall between two of the above-referenced values (e.g., a range of from about 14 minutes to about 94 hours, etc.).

In yet other non-limiting examples, the activated dendritic cell-containing composition may be administered after the period that the additional alternating electric field is applied has elapsed, wherein the activated dendritic cell-containing composition is administered within about 1 minute, about 5 minutes, about 10 minutes, about 15 minutes, about 30 minutes, about 45 minutes, about 1 hour, about 1.5 hours, about 2 hours, about 3 hours, about 4 hours, about 5 hours, about 6 hours, about 7 hours, about 8 hours, about 9 hours, about 10 hours, about 12 hours, about 15 hours, about 18 hours, about 21 hours, about 24 hours, about 27 hours, about 30 hours, about 33 hours, about 36 hours, about 39 hours, about 42 hours, about 45 hours, about 48 hours, about 51 hours, about 54 hours, about 57 hours, about 60 hours, about 63 hours, about 66 hours, about 69 hours, about 72 hours, about 75 hours, about 78 hours, about 81 hours, about 84 hours, about 87 hours, about 90 hours, about 93 hours, about 96 hours, and the like, of when the period of time elapsed.

The activated dendritic cell-containing composition(s) may be administered to the subject at any concentration that is capable of inducing an inflammatory response to the tumor or cancer cells. For example, but not by way of limitation, the activated dendritic cells may be administered at about 10 cells/kg body weight, about 100 cells/kg body weight, about 1000 cells/kg body weight, about 10⁴ cells/kg body weight, about 10⁵ cells/kg body weight, about 10⁶ cells/kg body weight, about 10⁷ cells/kg body weight, about 10⁸ cells/kg body weight, about 10⁹ cells/kg body weight, about 10¹⁰ cells/kg body weight, about 10¹¹ cells/kg body weight, about 10¹² cells/kg body weight, about 10¹³ cells/kg body weight, about 10¹⁴ cells/kg body weight, about 10¹⁵ cells/kg body weight, or higher, as well as a range formed from any of the above values (e.g., a range of from about 10⁴ to about 10⁹ cells/kg body weight, etc.).

In certain particular (but non-limiting) embodiments, the method involves concurrent therapy with two or more compositions. As such, the method may include an additional step of administering at least a second composition to the subject. Additional non-limiting examples of therapeutic agents that can be utilized as part of a second composition administered simultaneously or wholly or partially sequentially with the activated dendritic cell-containing composition include Lenvatinib, Pembrolizumab, and other anti-PD-1 therapeutics such as (but not limited to) Tislelizumab, Nivolumab, and Cemiplimab; an anti-LAG3 agent such as (but not limited to) OPDUALAG™ and/or Relatimab (Bristol-Myers Squibb, New York, NY); an anti-PD-L1 therapeutic agent, such as (but not limited to) Atezolizumab, Avelumab, and Durvalumab; an anti-CTLA-4 therapeutic agent, such as (but not limited to) Ipilimumab; chemotherapeutic agents, such as (but not limited to) Paclitaxel, Docetaxel, Ifosamide, Etoposide (Vepesid), Gemcitabine, Lomustine, Nab Paclitaxel, Temozolomide, and Carboplatin; TKI inhibitors, such as (but not limited to) Everolimus; mTOR inhibitors; Akt inhibitors; PI3K inhibitors; PARP inhibitors; VEGF inhibitors; FGF inhibitors; aromatase inhibitors (such as (but not limited to) Letrozole); biologics such as monoclonal antibodies (such as, but not limited to, Denosumab and Pembrolizumab); and the like, as well as any combinations thereof.

When present, the concurrent therapy may be performed substantially simultaneously or wholly or partially sequentially with the administration of the activated dendritic cell-containing composition. In addition, the two compositions may be administered via the same route (e.g., both orally administered or injected), or the two compositions may be administered by different routes (e.g., one composition orally administered and another composition intravenously administered).

When both the optional steps of administering a second composition and applying an alternating electric field to the subject to which the activated dendritic cell-containing composition has been administered are present, the optional administration step may be performed before or after the application of the alternating electric field has begun, and during application of the alternating electric field and/or after application of the alternating electric field has elapsed, in the same manner(s) and time frame(s) as described above for the antigen-loaded dendritic cell-containing composition.

That is, for example (but not by way of limitation), the second composition may be administered after application of the alternating electric field has commenced by a period of at least about 3 hours, about 6 hours, about 9 hours, about 12 hours, about 15 hours, about 18 hours, about 21 hours, about 24 hours, about 27 hours, about 30 hours, about 33 hours, about 36 hours, about 39 hours, about 42 hours, about 45 hours, about 48 hours, about 51 hours, about 54 hours, about 57 hours, about 60 hours, about 63 hours, about 66 hours, about 69 hours, about 72 hours, about 75 hours, about 78 hours, about 81 hours, about 84 hours, about 87 hours, about 90 hours, about 93 hours, about 96 hours, and the like, as well as a range formed from any of the above values (e.g., a range of from about 24 hours to about 96 hours, etc.), and a range that combines two integers that fall between two of the above-referenced values (e.g., a range of from about 14 hours to about 94 hours, etc.). In a particular (but non-limiting) embodiment, the second composition is administered at least about 24 hours after application of the alternating electric field has begun.

In other non-limiting examples, the second composition may be administered after the period of time that the alternating electric field is applied has elapsed, wherein the second composition is administered within about 3 hours, about 6 hours, about 9 hours, about 12 hours, about 15 hours, about 18 hours, about 21 hours, about 24 hours, about 27 hours, about 30 hours, about 33 hours, about 36 hours, about 39 hours, about 42 hours, about 45 hours, about 48 hours, about 51 hours, about 54 hours, about 57 hours, about 60 hours, about 63 hours, about 66 hours, about 69 hours, about 72 hours, about 75 hours, about 78 hours, about 81 hours, about 84 hours, about 87 hours, about 90 hours, about 93 hours, about 96 hours, and the like, of when the period of time elapsed. In a particular (but non-limiting) embodiment, the second composition is administered within about 96 hours of when the period of time elapsed.

In addition, for example (but not by way of limitation), the second composition may be administered after administration of the activated dendritic cell-containing composition by a period of at least about 1 minute, about 5 minutes, about 10 minutes, about 15 minutes, about 30 minutes, about 45 minutes, about 1 hour, about 2 hours, about 3 hours, about 4 hours, about 5 hours, about 6 hours, about 7 hours, about 8 hours, about 9 hours, about 10 hours, about 11 hours, about 12 hours, about 15 hours, about 18 hours, about 21 hours, about 24 hours, about 27 hours, about 30 hours, about 33 hours, about 36 hours, about 39 hours, about 42 hours, about 45 hours, about 48 hours, about 51 hours, about 54 hours, about 57 hours, about 60 hours, about 63 hours, about 66 hours, about 69 hours, about 72 hours, about 75 hours, about 78 hours, about 81 hours, about 84 hours, about 87 hours, about 90 hours, about 93 hours, about 96 hours, and the like, as well as a range formed from any of the above values (e.g., a range of from about 24 hours to about 96 hours, etc.), and a range that combines two integers that fall between two of the above-referenced values (e.g., a range of from about 14 hours to about 94 hours, etc.). In a particular (but non-limiting) embodiment, the second composition is administered at least about 12 hours after administration of the antigen-loaded dendritic cell-containing composition.

In certain particular (but non-limiting) embodiments, the method may further comprise the step of administering at least one additional therapy to the subject. Any therapies known in the art or otherwise contemplated herein for use with TTFields and/or activated dendritic cell therapy may be utilized in accordance with the methods of the present disclosure. Non-limiting examples of additional therapies that may be utilized include radiation therapy, photodynamic therapy, transarterial chemoembolization (TACE), or combinations thereof.

In certain particular (but non-limiting) embodiments, the method includes one or more additional steps. For example (but not by way of limitation), the method may further include repeating any of the steps one or more times. Each of the steps can be repeated as many times as necessary. When application of the alternating electric field is repeated, the transducer arrays may be placed in slightly different positions on the subject than their original placement; relocation of the arrays in this manner may further aid in treatment of the tumor/cancer. In addition, any of the administration steps (including the step of administering the dendritic cell-containing composition as well as any optional administration steps) may be repeated various times and at various intervals to follow any known and/or generally accepted dosage/treatment regimen for the composition(s)/therapy(ies).

While the methods described herein above are related to use of the activated dendritic cells in cancer treatment, it will be understood that the scope of the present disclosure is not limited to use in cancer treatment. Rather, the present disclosure encompasses activation of dendritic cells via exposure to an alternating electric field followed by loading of the subsequently activated dendritic cells with any desired antigens for treatment of any other related diseases, infections, or conditions for which dendritic cell therapy is beneficial. For example, but not by way of limitation, the dendritic cells activated by exposure to alternating electric fields may be pulsed with antigens (or co-cultured with a source of antigens) that include (but are not limited to) bacterial, viral, fungal, parasitic, tumor, cancer antigens, and the like, as well as any combinations thereof.

Certain non-limiting embodiments of the present disclosure are related to immunogenic compositions produced by any of the methods disclosed or otherwise contemplated herein.

Certain non-limiting embodiments of the present disclosure are related to immunogenic compositions that comprise a population of isolated, antigen-loaded dendritic cells, wherein the antigen-loaded dendritic cells are produced by co-culturing dendritic cells with at least one cancer cell isolated from a subject to produce the antigen-loaded dendritic cells, and wherein the at least one cancer cell has been exposed to an alternating electric field in vivo or ex vivo prior to co-culture with the dendritic cells.

Certain non-limiting embodiments of the present disclosure are related to immunogenic compositions that comprise a population of any of the isolated, antigen-loaded (and optionally alternating electric field-exposed) dendritic cells produced as described or otherwise contemplated herein. In certain particular (but non-limiting) embodiments, the dendritic cells have further been co-cultured with at least one cancer cell isolated from a subject to produce activated, antigen-loaded dendritic cells. In a particular (but non-limiting) embodiment, the TTFields may have been applied to a subject or to either or both cell types prior to or during the co-culture step, so that the dendritic cells and/or the cancer cell(s) utilized in the co-culture have been exposed to an alternating electric field ex vivo. In yet another particular (but non-limiting) embodiment, the dendritic cells have been co-cultured or pulsed for loading of other types of antigens in the dendritic cells.

The immunogenic composition may be formulated for administration by any of the administration routes disclosed or otherwise contemplated herein. In certain particular (but non-limiting) embodiments, the immunogenic composition is formulated for intradermal, subcutaneous, intravenous, and/or intranodal administration.

In certain particular (but non-limiting) embodiments, the immunogenic composition may further include one or more additional active agents that further assists in stimulating the immune system to recognize and attack the cancer cells (or other diseased, infected, or bacterial cells) in the subject. Non-limiting examples of additional agents that may be present in the immunogenic composition include an adjuvant, a cytokine, an interferon, a TLR agonist, a STING (stimulator of interferon genes) agonist, GM-CSF, CD40L, Fms related tyrosine kinase 3 ligand (FLT3L), a C type Lectin Receptor (CLR), an anti-LAG3 agent (such as, but not limited to, OPDUALAG™ and/or Relatimab (Bristol-Myers Squibb, New York, NY)), and combinations thereof.

The dendritic cells of the compositions and methods may include any dendritic cells known in the art or otherwise contemplated herein. For example, but not by way of limitation, the dendritic cells may comprise at least one of conventional DC 1 (cDC1), cDC2, plasmacytoid DC (pDC), and the like.

Certain non-limiting embodiments of the present disclosure are related to kits that include any of the components of the alternating electric field-generating systems (such as, but not limited to, one or more transducer arrays and/or one or more hydrogel compositions, as disclosed in U.S. Pat. Nos. 7,016,725; 7,089,054; 7,333,852; 7,565,205; 8,244,345; 8,715,203; 8,764,675; 10,188,851; and 10,441,776; and in US Patent Application Nos. US 2018/0160933; US 2019/0117956; US 2019/0307781; and US 2019/0308016) in combination with any component, devices, and/or reagents utilized in one or more of the steps of isolating one or more cell types (e.g., dendritic cells or precursors thereof and/or cancer cells), exposing the subject, the dendritic cells (or precursors thereof), and/or the cancer cells to the TTFields, co-culturing the dendritic cells and cancer cells, pulsing the dendritic cells with antigens, isolating the activated dendritic cells, and/or formulating the activated dendritic cells for administration to the subject, in accordance with the methods disclosed or otherwise contemplated herein. The kits may optionally further include one or more of any of the optional compositions disclosed or otherwise contemplated herein (such as, but not limited to, one or more compositions utilized in an optional concurrent therapy step(s)). The kits may optionally further include one or more devices (or one or more components of devices) utilized in one or more additional therapy steps.

In a particular (but non-limiting) embodiment, the kit may further include instructions for performing any of the methods disclosed or otherwise contemplated herein. For example (but not by way of limitation), the kit may include instructions for isolating one or more cell types, exposing a subject and/or a cell culture to the alternating electric field-generating system, instructions for isolating the activated dendritic cells and formulating for administration to a subject, instructions for applying one or more components of the alternating electric field-generating system to the skin of the subject, instructions for applying the alternating electric field to the subject, instructions for when and how to administer the dendritic cell-containing composition(s) and optionally how to administer one or more optional additional compositions, and/or instructions for when to activate and turn off the alternating electric field in relation to the administration of the dendritic cell-containing composition(s) and/or administration of one or more optional compositions.

In addition to the components described in detail herein above, the kits may further contain other component(s)/reagent(s) for performing any of the particular methods described or otherwise contemplated herein. For example (but not by way of limitation), the kits may additionally include: (i) components for preparing the skin prior to disposal of the hydrogel compositions and/or transducer arrays thereon (e.g., a razor, a cleansing composition or wipe/towel, etc.); (ii) components for removal of the gel/transducer array(s); (iii) components for cleansing of the skin after removal of the gel/transducer array(s); (iv) components for isolation of cancer cells/portion of tumor; (v) components for isolation of dendritic cells or precursors thereof; and/or (vi) components for maturation of the dendritic cells or precursors thereof. The nature of these additional component(s)/reagent(s) will depend upon the particular treatment format, and identification thereof is well within the skill of one of ordinary skill in the art; therefore, no further description thereof is deemed necessary. Also, the components/reagents present in the kits may each be in separate containers/compartments, or various components/reagents can be combined in one or more containers/compartments, depending on the sterility, cross-reactivity, and stability of the components/reagents.

The kit may be disposed in any packaging that allows the components present therein to function in accordance with the present disclosure. In certain non-limiting embodiments, the kit further comprises a sealed packaging in which the components are disposed. In certain particular (but non-limiting) embodiments, the sealed packaging is substantially impermeable to air and/or substantially impermeable to light.

In addition, the kit can further include a set of written instructions explaining how to use one or more components of the kit. A kit of this nature can be used in any of the methods described or otherwise contemplated herein.

In certain non-limiting embodiments, the kit has a shelf life of at least about six months, such as (but not limited to), at least about nine months, or at least about 12 months.

Certain non-limiting embodiments of the present disclosure are related to systems that include any of the components of the alternating electric field generating systems (such as, but not limited to, one or more transducer arrays and/or one or more hydrogel compositions, as disclosed in U.S. Pat. Nos. 7,016,725; 7,089,054; 7,333,852; 7,565,205; 8,244,345; 8,715,203; 8,764,675; 10,188,851; and 10,441,776; and in US Patent Application Nos. US 2018/0160933; US 2019/0117956; US 2019/0307781; and US 2019/0308016) in combination with at least one of any of the compositions comprising activated dendritic cells disclosed or otherwise contemplated herein. The systems may optionally further include one or more of any of the optional compositions disclosed or otherwise contemplated herein. The systems may optionally further include one or more devices (or one or more components of devices) utilized in the various isolation, co-culture, or administration steps, or optional additional treatment/therapy steps.

### EXAMPLES

Examples are provided herein below. However, the present disclosure is to be understood to not be limited in its application to the specific experimentation, results, and laboratory procedures disclosed herein after. Rather, the Examples are simply provided as one of various embodiments and is meant to be exemplary, not exhaustive.

### Example 1

### Summary:

Dendritic cells (DC) are critical components of the cancer-immunity cycle, driving T cells to activation or tolerance [Chen DS, Immunity, 2013]. The effects of TTFields on human DC is yet unknown but TTFields' mode of action may plausibly affect DCs' viability or their ability to undergo activation. After we had tested the effects of TTFields on human monocyte derived DC (IL4+GMCSF cultured monocytes) we transitioned to physiological blood-borne DC: conventional DC 1 (cDC1), cDC2, and plasmacytoid DC (pDC).

The effects of TTFields on the viability and the ability of the DC to undergo activation and maturation were tested. As differences were in some cases subtle, and as the INOVITRO™ system (Novocure GmbH, Root, Switzerland) may introduce considerable intra-experimental variability, 8 experiments were performed to confirm the results. The TTFields conditions utilized were 150 and 200 kHz, as those approved for treatment of lung and brain cancers respectively. Field intensity was 2.7V/cm and exposure time was 48 hours (DC substantially change in culture beyond 2 days). We used freshly purified peripheral blood mononuclear cells (PBMC) obtained from heathy blood donors. The PBMC were evaluated on Day 0—to define the DC baseline status (group 1). Following 48 h of culture additional 6 groups were monitored: (group 2) control—TTFields-untreated DC grown in INOVITRO™ dishes (3) control+LPS+R848 (denoted henceforth LPS)—full activation under standard growth conditions (4) 150 kHz TTFields—evaluating the effects of TTFields on DC viability and maturation (5) 150 kHz TTFields+LPS evaluating the effects of activatory signals under TTFields conditions (6) 200 kHz TTFields, and (7) 200 kHz TTFields+LPS.

Following treatment, the cells were stained by a 12-color DC panel, identifying all 3 physiological DC subsets, 2 maturation/activation markers, and cell viability. Analysis of the 8 experiments revealed that in the TTFields conditions induced a slight, usually non-significant reduction (2-6%) in viability. Importantly, it was repeatedly seen that the 150 kHz frequency drove potent DC activation compared to the 2d TTFields-untreated control. The TTFields 200 kHz frequency did not induce this activation. Activation with strong DC activator, LPS, even under TTFields conditions further activated the DC, demonstrating that physiological DC can undergo effective activation under TTFields.

These results demonstrate that physiological DC of all subtypes retain their viability and their capacity to mature—two parameters critical for effective DC function. These results are also indicative that TTFields, at specific frequencies, may serve as a “physical adjuvant” capable of driving DC maturation.

Experimental design: A series of 8 experiments (15 technical repeats per each group in total) was performed in order to assess the effect of TTFields treatment on the viability and the potential for activation of blood-borne physiological dendritic cells. TTFields' frequencies used were 150 kHz and 200 kHz, 2.7 V/cm field intensity for 48 hours. Fresh PBMC from healthy donors were split to 7 groups as described in Table 1. DC activation was induced by the exposure of cultures to 1 ug/mL lipopolysaccharides from E. coli strain O111:B4+2.5 ug/mL R848 (Resiquimod) (denoted—LPS). Following 48 hours of treatment, cells were stained with a 12 color flow cytometric panel (Table 2). Nine of the channels were used to identify the three DC subtypes, one channel was used to distinguish live from dead cells and two channels were used to quantify the expression of CD80 and CD83—DC maturation/activation markers. The stained cells were read on a 4-laser BD FACSAria-Fusion flow cytometer and analyzed using the FlowJo package.

TABLE 1
Treatment Groups in the Performed Experiments.

| Group | Group Name | Group Purpose |
|---|---|---|
| 1 | Day-0 | Negative control, the cells in the blood at time 0 |
| 2 | Control (d2) | Control, the background of the experiment |
| 3 | Control + LPS (d2) | Positive control - the maximal activation of the cells |
| 4 | 150 kHz TTFields (d2) | Assessment of 150 kHz effect on DC |
| 5 | 150 kHz TTFields + LPS (d2) | The 150 kHz interaction with LPS - DC viability and activation |
| 6 | 200 kHz TTFields (d2) | Assessment of 200 kHz effect on DC |
| 7 | 200 kHz TTFields + LPS (d2) | The 200 kHz interaction with LPS - DC viability and activation |

TABLE 2
List of Markers and Clones in the DC Flow Cytometric Panel.

| Channel | Antigen/s | Clone | Manufacturer |
|---|---|---|---|
| 1 | VivaFix | NA | BioRad |
| 2 | CD14 | M5E2 | BD |
| 3 | CD141 | 1A4 | BD |
| 4 | CD16 | 3G8 | BD |
| 5 | CD19 | 555414 | BD |
| 6 | CD83 | HB15e | BD |
| 7 | CD56 | NCAM16.2 | BD |
| Lineage | CD3 | SK7 | BD |
| channel | CD66b | G10F5 | BD |
| 8 | CD45 | 2D1 | BioLegend |
| 9 | CD1c | L161 | BioLegend |
| 10 | CD123 | 6H6 | BioLegend |
| 11 | HLA-DR | LN3 | Invitrogen |
| 12 | CD80 | 2D10.4 | Invitrogen |

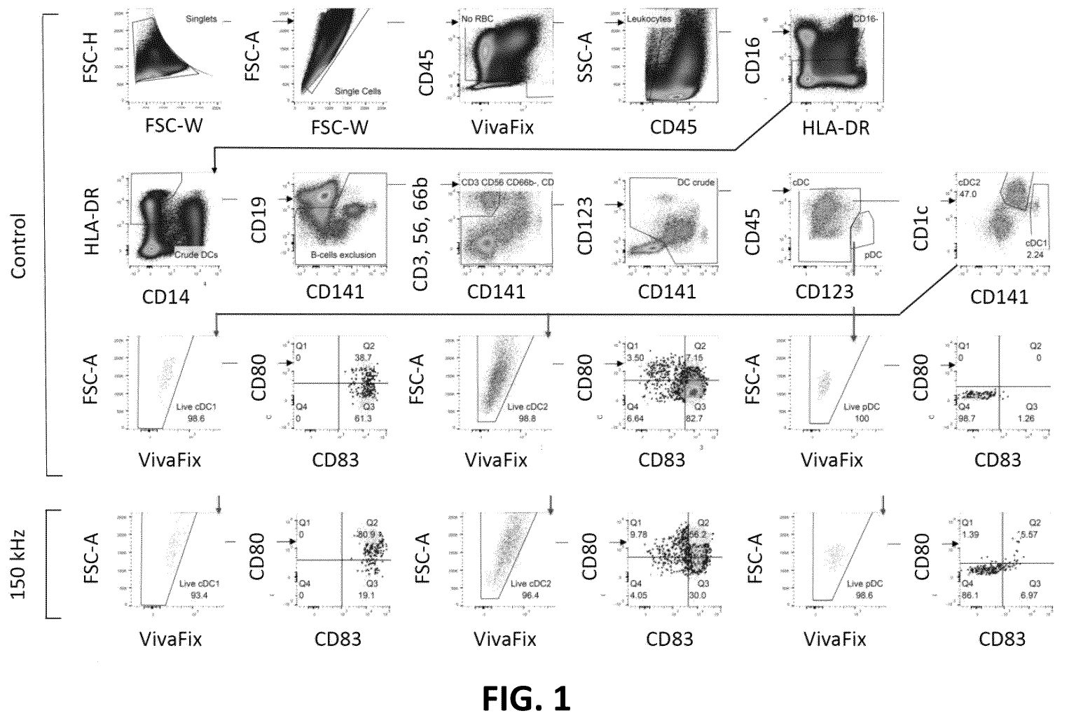

A gating strategy was devised that was aimed at assessing the viability and the maturation of the three blood DC subtypes (FIG. 1). The figure demonstrates full gating of the control group, and the bottom two rows demonstrate the viability and activation of the control group and that of the TTFields 150 kHz group, depicting the three DC subtypes in each row.

In most of the experiments performed, technical repeats were done (15 repeats per 8 experiments). This was done as the INOVITRO™ system is noisy, especially when querying cells from PBMC of different donors where the cells are infrequent (0.03%-0.5%) and sensitive to minor changes in culture. The results presented per each experiment are the mean values in the corresponding experiment (1-3 technical repeats per group). In each experiment, approximately 3.5×10⁶ PBMCs were cultured per each repeat. Paired t-test was used to test the significance per the eight summed experiments.

### Results:

### DC Viability is Grossly Retained Under TTFields

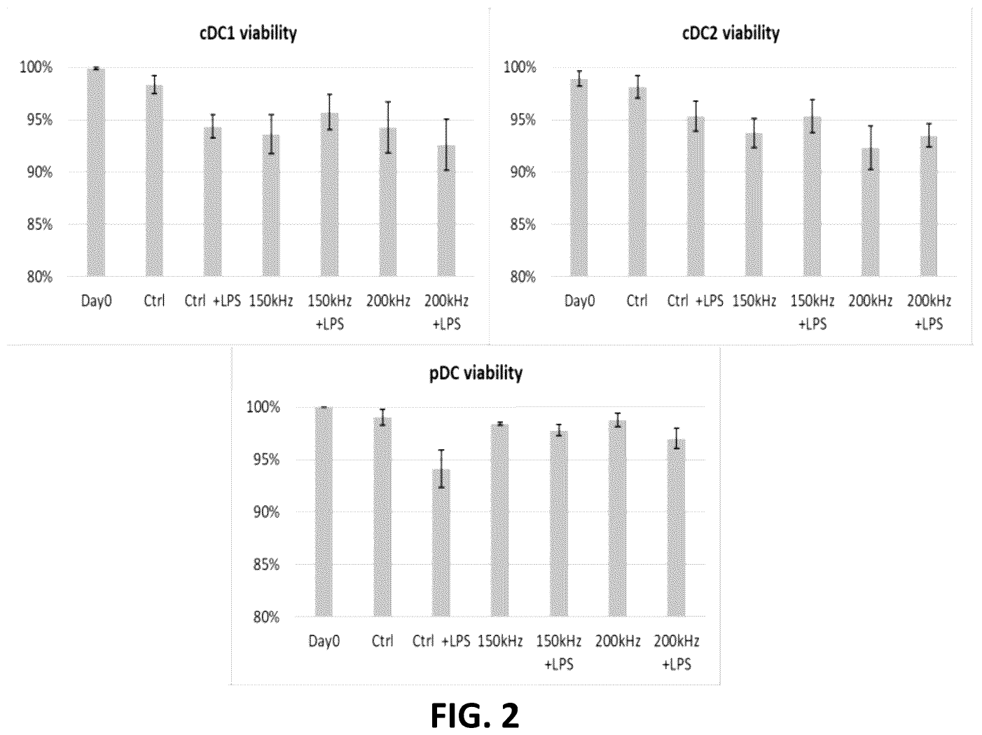

FIG. 2 shows cross experiment mean viability±SEM for the various groups. With a bar-graph scale ranging from 80-100%, only minor differences in viability can be noted. Overall, the differences in viability among the non-LPS treated groups to day-2 controls were in the range of 2-6%: cDC1-4% mean reduced viability under TTFields (NS), cDC2-4% mean reduced viability (P=0.03) for 150 kHz and 6% mean reduced viability for 200 kHz (NS). For the pDCs treated with 150 kHz there was a 2% reduction in viability (P=0.01). While some of the means were significantly reduced, the effect size in all groups was very small. LPS, in all groups, reduced DC viability, yet there were no statistical differences between the TTFields+LPS groups to the control+LPS groups.

### The Effect of TTFields on DC Activation and Maturation

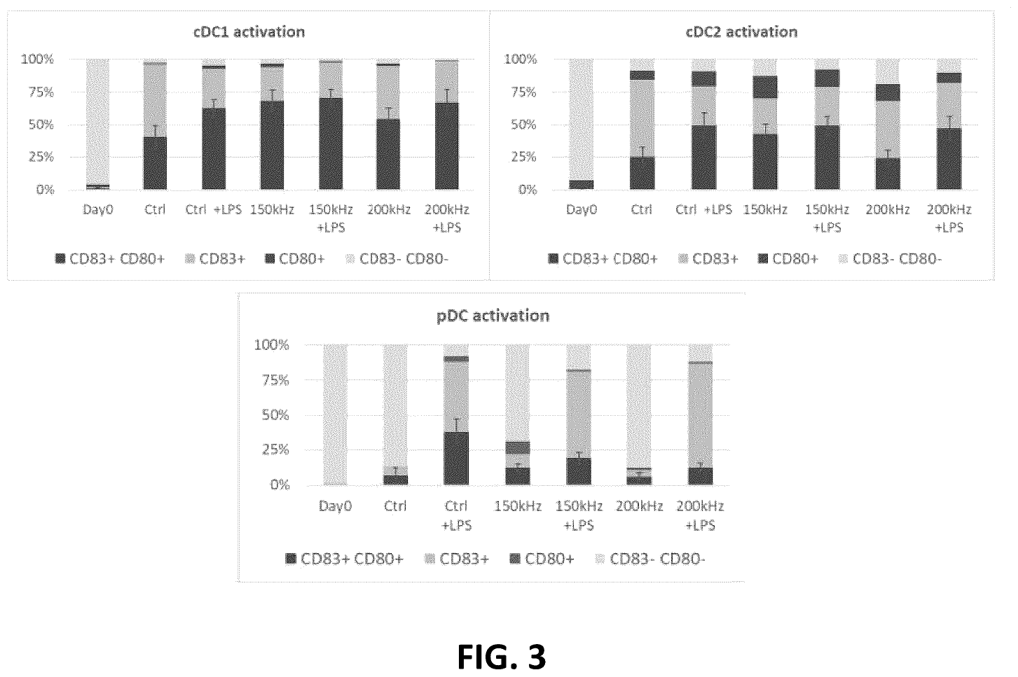

Next, the effects of TTFields were tested on DC maturation and on their capacity to undergo activation driven by TLR-receptor agonists: TLR4 agonist—LPS and TLR7/8 agonist—R848. The combination of R848 and LPS provides strong stimulation for all physiological DC [Lovgren T, Cancer Imm. Immunother, 2017]. FIG. 3 depicts the average±SEM of the two monitored maturation markers—CD80 (B7.1) and CD83, either singly expressed (e.g., CD83+CD80−) or co-expressed—CD83+CD80+. Double positive cells represent fully mature DC [Dudek A M, front immu 2013].

For all the DC types, maturation at day 0 was close to zero. By day 2 (48 hours), the two cDC subtypes and to a lesser extent pDC subtype exhibited increased expression of CD80 and/or CD83. The single positive increase in the cDC group was mainly driven by the increase in the expression of the CD83. For all DC subtypes, exposure to LPS increased the fraction of double positive DC.

The Effect of TTFields on cDC1 Dendritic Cells

CDC1 are the most infrequent dendritic cells in human (3%-5% of DC), but are critical components for immune rejection of tumors. Uniquely, they can collect dead cell material and cross present it on MHC-I to cytotoxic T cells [Wculek S K, Nat Rev Immunol 2020, Volovitz I, 2016 Int Rev Immunol]. As for cDC1 activation (Table 3), all treated groups, but 200 kHz, exhibited significantly higher fractions of fully-activated (double positive) DC. This increase in double positive DC was detected also in the 150 kHz treated group without LPS (P=0.003); 200 kHz treatment did not induce an increase in the double positive DC fraction compared to control. The 150+LPS and 200+LPS exhibited similar double positive fractions as the control (all ranging from 63%-70%), indicating that cDC1 can undergo effective activation under TTFields. While 200 kHz+LPS was significantly higher in double positives than 200 kHz, 150 kHz+LPS was only slightly higher (NS) than the 150 kHz. Of the total increase in double positive cDC1 (total increase=subtracting 150 kHz+LPS From day-2 control), 93% could be achieved due to the exposure of cDC1 to TTFields 150 kHz.

TABLE 3
Individual and summed results of cDC1 double positive
fully mature DC fractions (CD80+, CD83+).

| | | | | 150 kHz | LPS + | | LPS + |
|---|---|---|---|---|---|---|---|
| | Day 0 | Control | LPS | TTF | 150 kHz | 200 kHz | 200 kHz |
| Exp. 1 | 0.00 | 0.19 | 0.45 | 0.46 | 0.38 | NA | NA |
| Exp. 2 | 0.00 | 0.23 | 0.40 | 0.70 | 0.69 | 0.62 | 0.78 |
| Exp. 3 | 0.00 | 0.75 | 0.61 | 0.87 | 0.90 | 0.65 | NA |
| Exp. 4 | 0.00 | 0.26 | 0.72 | 0.80 | 0.51 | 0.65 | 0.68 |
| Exp. 5 | 0.05 | 0.36 | 0.66 | 0.77 | 0.71 | 0.54 | 0.74 |
| Exp. 6 | 0.01 | 0.76 | 0.88 | 0.97 | 0.88 | 0.76 | 0.94 |
| Exp. 7 | 0.01 | 0.20 | 0.51 | 0.33 | 0.67 | 0.14 | 0.30 |
| Exp. 8 | 0.02 | 0.50 | 0.80 | 0.57 | 0.89 | 0.46 | 0.59 |
| Mean | 0.01 | 0.41 | 0.63 | 0.68 | 0.70 | 0.55 | 0.67 |
| Sig. vs control (P-value) | 0.002 | ←NA→ | 0.01 | 0.003 | 0.0005 | 0.22 | 0.014 |
| Sig. group-A vs group-A + | NA | NA → | 0.01 | NA → | 0.798 | NA → | 0.002 |
| LPS (P-value) | | | | | | | |

The fractions of double positive DC on day-0 and after 48 hours of culture. Shown are either individual fractions or the mean of 2-3 technical repeats per each treated group. Means per 8 experiments are given, below which there are two rows of statistical comparisons - the top row compares all groups to the day-2 control (TTFields untreated). The bottom row compares each treated groups to the same groups treated with LPS, e.g. 150 kHz to 150 kHz + LPS. NA in the fractions of responding cells denotes experiments where the specific DC subset could not be unequivocally identified using the gating strategy.

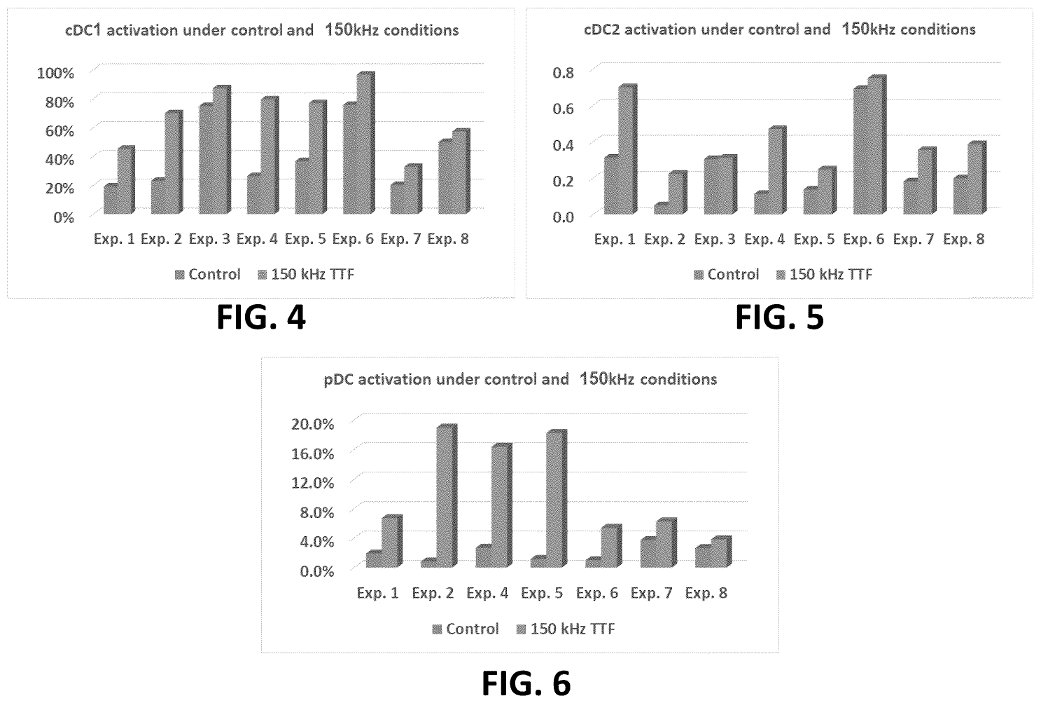

To illustrate the effect of 150 kHz on cDC1, FIG. 4 shows a comparison of the cDC1 double positive cells (CD80+ and CD83+) in the control and in the 150 kHz groups across all 8 experiments.

The Effect of TTF on cDC2-Type Dendritic Cells

CDC2 are the most common conventional DC and are the only DC subtype found in glioblastoma in non-negligible numbers. CDC2 can effectively activate helper T cells. They secrete higher amounts of inflammatory cytokines as IL1β, IL6, TNFα, and IL8 than cDC1. They may serve pro- or anti-tumoral roles within tumors depending on the context of their activation and their maturation status [Wculek S K, Nat Rev Immunol 2020, Volovitz I, Int Rev Immunol, 2016].

Table 4 shows that the activation of cDC2 mirrored that of cDC1 in many aspects. Here too, all treated groups, but the 200 kHz show significantly higher double positive, fully-activated, DC than the day-2 control. Exposure of cDC2 to 150 kHz without LPS significantly enhanced the fraction of double positive cDC2 (P=0.006) while 200 kHz had no effect on the fraction of double positive cDC2. The 150+LPS and 200+LPS showed a similar double-positive fraction as the control (all ranging from 48%-50%) demonstrating that cDC2 can effectively undergo activation under TTFields. While the double positive fraction in 200 kHz+LPS was significantly higher in double positives than 200 kHz, 150 kHz+LPS was only slightly higher (NS) than 150 kHz. The 150 kHz frequency had induced 72% of the total achievable increase in double positive cDC2 (subtracting 150 kHz+LPS from day-2 control).

TABLE 4
Individual and summed results of cDC2 double-positive
fully mature DC fractions (CD80+, CD83+)

| | | | | 150 kHz | LPS + | | LPS + |
|---|---|---|---|---|---|---|---|
| | Day 0 | Control | LPS | TTF | 150 kHz | 200 kHz | 200 kHz |
| Exp. 1 | 0 | 0.31 | 0.52 | 0.70 | 0.44 | NA | NA |
| Exp. 2 | 0 | 0.05 | 0.18 | 0.22 | 0.18 | 0.09 | 0.16 |
| Exp. 3 | 0.01 | 0.30 | 0.16 | 0.31 | 0.39 | 0.21 | NA |
| Exp. 4 | 0 | 0.11 | 0.60 | 0.47 | 0.43 | 0.29 | 0.47 |
| Exp. 5 | 0 | 0.14 | 0.30 | 0.25 | 0.48 | 0.18 | 0.48 |
| Exp. 6 | 0.01 | 0.69 | 0.84 | 0.75 | 0.76 | 0.52 | 0.78 |
| Exp. 7 | 0 | 0.18 | 0.67 | 0.35 | 0.62 | 0.19 | 0.44 |
| Exp. 8 | 0 | 0.20 | 0.70 | 0.39 | 0.66 | 0.22 | 0.51 |
| Mean | 0 | 0.25 | 0.50 | 0.43 | 0.50 | 0.24 | 0.48 |
| Sig. vs control (P-value) | 0.01 | ←NA→ | 0.018 | 0.006 | 0.003 | 0.9350 | 0.004 |
| Sig. group-A vs group-A + | NA | NA → | 0.018 | NA → | 0.3559 | NA → | 0.0014 |
| LPS (P-value) | | | | | | | |

The fractions of double positive cDC2 on day-0 and after 48 hours of culture. Shown are either individual fractions or the mean of 2-3 technical repeats per each treated group. Means per 8 experiments are given, below which there are two rows of statistical comparisons - the top row compares all groups to the day-2 control (TTFields untreated). The bottom row compares each treated groups to the same groups treated with LPS, e.g., 150 kHz to 150 kHz + LPS. NA in the fractions of responding cells denotes experiments where the specific DC subset could not be unequivocally identified using the gating strategy.

To illustrate the effect of 150 kHz TTF on cDC2 activation, FIG. 5 shows a comparison between the double positive cells in the control (day 2) and 150 kHz.

### The Effect of TTF on pDC

PDC are the main cellular producers of type-1 IFNs (IFN-α/β) in an early response to viruses, bacteria or self nucleic acids. IFN-α is an important antiviral and antitumoral immune factor. [Wculek S K, Nat Rev Immunol 2020, Volovitz I, 2016 Int Rev Immunol]. Table 5 shows that TTFields induced a relatively smaller effect on the fraction of double positive pDC than on cDC. In the 150 kHz the mean double positive cells fraction was significantly higher than control (P=0.018) yet only 11% of cells were double positive in this group compared to 2% in the control (9% difference). Again, here the 200 kHz had no effect of DC maturation. The activation to LPS was consistently higher without TTFields (mean 35%) than under 150 kHz (mean 18%) or 200 kHz (mean 12%) yet these differences did not reach statistical significance. Here again, 200 kHz+LPS was significantly higher than 200 kHz, and 150 kHz+LPS was non-significantly higher than 150 kHz. Here, only 56% of the increase in double positive pDC could be induced by the 150 kHz treatment.

TABLE 5
Individual and summed results of pDC double-positive fully-mature DC fractions (CD80+, CD83+)
pDC Activation

| | | | | 150 kHz | LPS + | | LPS + |
|---|---|---|---|---|---|---|---|
| | Day 0 | Control | LPS | TTF | 150 kHz | 200 kHz | 200 kHz |
| Exp. 1 | 0.00 | 0.02 | 0.03 | 0.07 | 0.04 | NA | NA |
| Exp. 2 | 0.00 | 0.01 | 0.11 | 0.19 | 0.16 | 0.02 | 0.09 |
| Exp. 3 | NA | NA | NA | NA | NA | NA | NA |
| Exp. 4 | 0.00 | 0.03 | 0.61 | 0.16 | 0.10 | 0.03 | 0.11 |
| Exp. 5 | 0.00 | 0.01 | 0.47 | 0.18 | 0.30 | 0.03 | 0.11 |
| Exp. 6 | 0.00 | 0.01 | 0.11 | 0.05 | 0.08 | 0.07 | 0.25 |
| Exp. 7 | 0.00 | 0.04 | 0.46 | 0.06 | 0.23 | 0.02 | 0.09 |
| Exp. 8 | 0.00 | 0.03 | 0.70 | 0.04 | 0.31 | 0.03 | 0.08 |
| Mean | 0.00 | 0.02 | 0.35 | 0.11 | 0.18 | 0.03 | 0.12 |
| Sig. vs control (P-value) | 0.003 | ←NA→ | 0.015 | 0.018 | 0.008 | 0.2761 | 0.018 |
| Sig. group-A vs group-A + | NA | NA → | 0.015 | NA → | 0.199 | NA → | 0.005 |
| LPS (P-value) | | | | | | | |

The fractions of double positive pDC on day-0 and after 48 hours of culture. Shown are either individual fractions or the mean of 2-3 technical repeats per each treated group. Means per 8 experiments, are given, below which there are two rows of statistical comparisons - the top row compares all groups to the day-2 control (TTFields untreated). The bottom row compares each treated groups to the same groups treated with LPS, e.g. 150 kHz to 150 kHz + LPS. NA in the fractions of responding cells denotes experiments where the specific DC subset could not be unequivocally identified using the gating strategy.

To illustrate the effects of 150 kHz TTF on pDC double positive cells, FIG. 6 shows the mean cell frequencies from the controls and the matching 150 kHz samples. While the scales for activation are lower in the pDC than in the cDC, a consistent pDC activation driven by 150 kHz can be noted in all experiments.

### Conclusions

Only minor differences were found in the viability of DC treated with TTFields. Also, significant differences were not found between the fraction of double positive (CD80+CD83+) DC between the control+LPS and the TTFields-treated samples+LPS, indicating that TTFields does not suppress DC-maturation by activatory reagents as LPS+R848.

The data from 8 experiments indicates that physiological DCs cDC1, cDC2, and pDC are activated by the 150 kHz TTFields treatment. In fact, for all DC subtypes, the major part of the maturation achievable by exposure to a strong activator such as LPS could also be achieved solely by the exposure of PBMC to TTFields at 150 kHz. These results indicate that TTFields may act as a “physical adjuvant” that enhances the maturation status of DC in an antigen-non-specific manner.

DC serve critical roles within tumors, by attracting T cells to the tumor area and within it and then reactivating these T cells to enable their effective anti-tumoral responses [Wculek S K, Nat Rev Immunol 2020]. The immunostimulatory effects of 150 kHz may drive tumoral DC to fully mature even after only 48 hours of exposure. DC maturation is a critical parameter as to the ability of DC to drive potent anti-tumoral responses. The differences between TTFields treatment with 150 kHz versus 200 kHz may not only affect the potential to kill specific tumor cells. It may, as we have previously shown, affect the viability and function of tumor-infiltrating T cells [Diamant G, 2021, J Immunol], or affect the maturation status or the function of DC within the treated range.

### Example 2

The primary goal of cancer immunotherapy is to activate a preexisting, endogenous immune response in cancer patients. Although significant advances have been made in this field, treatment efficacy still needs to be improved. A personalized cancer vaccine is a promising strategy to strengthen the anti-tumor immune response via an immunogenic form of apoptosis, also known as immunogenic cell death (ICD). ICD is characterized by the emission of danger-associated molecular patterns that serve to recruit immune cells to the site of the tumor. Previous studies have shown that TTFields treatment potentiates immunogenic cell death in cancer cells, ultimately stimulating an immune response through engulfing cancer cells. In turn, it presents neoantigens to initiate adaptive immunity further.

To support the rationale for using TTFields as an immune modulator, mice are treated with TTFields for 72 h using the INOVITRO™ system (Novocure GmbH, Root, Switzerland). Cancer cells are then isolated from the mice. PBMCs are also isolated from the mice, either before or after TTFields exposure, or from an HLA-matched donor. The PBMCs are co-cultured with the cancer cells to activate and load neoantigens into the dendritic cells. The activated, antigen-loaded dendritic cells are isolated away from the co-culture and the cancer cells and then administered to mice as a vaccine to trigger an immune response to cancer development.

In this manner, methods of increasing immunity to cancer cells are combined with TTFields treatment. The combination of TTFields treatment with administration of personalized activated dendritic cell-containing composition(s) provides a synergistic effect over either treatment alone and initiates an immune response in the patient that will allow the immune system to eliminate the cancer cells.

### Example 3

Dendritic cells are activated by exposure to TTFields as in Example 1. The TTFields-exposed dendritic cells are then antigen-loaded by pulsing with antigens of interest or co-culture with a source of antigen (e.g., cancer cells, bacterial cells, viral-infected cells, fungal-infected cells, etc.). The activated, antigen-loaded dendritic cells are isolated away from the culture and then administered to the same subject or an allogenic subject as an immunomodulator or vaccine to trigger an immune response. In this manner, TTFields treatment acts as a physical adjuvant.

### NON-LIMITING ILLUSTRATIVE EMBODIMENTS OF THE INVENTIVE CONCEPT(S)

Illustrative embodiment 1. A method of activating dendritic cells, the method comprising the step of: applying an alternating electric field to a composition comprising immature dendritic cells or precursors thereof in vitro for a period of time sufficient to produce activated dendritic cells.

Illustrative embodiment 2. The method of illustrative embodiment 1, further comprising the step of contacting the activated dendritic cells with a source of antigens to produce antigen-loaded dendritic cells.

Illustrative embodiment 3. A method of preparing an immunogenic composition, the method comprising the steps of: applying an alternating electric field to a composition comprising immature dendritic cells or precursors thereof in vitro for a period of time sufficient to produce activated dendritic cells; contacting the activated dendritic cells with a source of antigens to produce antigen-loaded dendritic cells; and isolating the antigen-loaded dendritic cells to form the immunogenic composition.

Illustrative embodiment 4. The method of illustrative embodiment 2 or 3, wherein the dendritic cells are pulsed with antigens.

Illustrative embodiment 5. The method of illustrative embodiment 2 or 3, wherein the dendritic cells are co-cultured with a source of antigens.

Illustrative embodiment 6. The method of illustrative embodiment 5, wherein the contacting step is further defined as co-culturing the mature dendritic cells with at least one cancer cell isolated from the subject to produce antigen-loaded dendritic cells.

Illustrative embodiment 7. The method of any of illustrative embodiments 2-6, wherein the antigens are selected from the group consisting of bacterial, viral, fungal, tumor, and cancer antigens.

Illustrative embodiment 8. A method of preparing an immunogenic composition, the method comprising the steps of: (1) applying an alternating electric field ex vivo to a composition comprising immature dendritic cells and/or dendritic cell precursors to produce mature dendritic cells; (2) co-culturing the mature dendritic cells with at least one cancer cell isolated from the subject to produce antigen-loaded dendritic cells; and (3) isolating the antigen-loaded dendritic cells from the co-culture of (2) and from the at least one cancer cell to form the immunogenic composition.

Illustrative embodiment 9. A method of treating cancer in a subject, the method comprising the steps of: (1) applying an alternating electric field ex vivo to a composition comprising immature dendritic cells and/or dendritic cell precursors to produce mature dendritic cells; (2) co-culturing the mature dendritic cells with at least one cancer cell isolated from the subject to produce antigen-loaded dendritic cells; (3) isolating the antigen-loaded dendritic cells from the co-culture of (2) and from the at least one cancer cell; and (4) administering the antigen-loaded dendritic cells to the subject.

Illustrative embodiment 10. The method of illustrative embodiment 8 or 9, wherein at least a portion of steps (1) and (2) are performed simultaneously, whereby the alternating electric field is also applied to the at least one cancer cell during the co-culture.

Illustrative embodiment 11. The method of any of illustrative embodiments 8-10, wherein the at least one isolated cancer cell is exposed to an alternating electric field prior to step (2).

Illustrative embodiment 12. The method of illustrative embodiment 11, wherein an alternating electric field is applied to a target region of the subject prior to isolation of the at least one cancer cell from the subject.

Illustrative embodiment 13. The method of illustrative embodiment 11 or 12, wherein the at least one cancer cell is exposed to an alternating electric field ex vivo and prior to co-culture.

Illustrative embodiment 14. The method of any of illustrative embodiments 8-13, wherein the method comprises isolating the composition from the subject prior to step (1).

Illustrative embodiment 15. The method of illustrative embodiment 14, wherein the composition comprises peripheral blood mononuclear cells (PBMCs).

Illustrative embodiment 16. The method of illustrative embodiment 14 or 15, wherein isolation of the composition is further defined as comprising the steps of: isolating immature monocytes (dendritic cell precursors) from the blood stream of the subject; and generating immature dendritic cells from the immature monocytes/dendritic cell precursors.

Illustrative embodiment 17. The method of any of illustrative embodiments 8-16, wherein the composition of (1) comprises PBMCs isolated from an HLA-matched donor. Illustrative embodiment 18. The method of any of illustrative embodiments 8-17, wherein the at least one cancer cell is further defined as at least a portion of a solid tumor.

Illustrative embodiment 19. The method of any of illustrative embodiments 8-18, wherein step (2) is performed in the presence of at least one composition selected from the group consisting of a cytokine, an interferon, granulocyte-macrophage colony-stimulating factor (GM-CSF), CD40 ligand (CD40L), a Toll-like receptor (TLR) agonist, and combinations thereof.

Illustrative embodiment 20. The method of any of illustrative embodiments 9-19, further comprising the step of: (5) applying the alternating electric field to the target region of the subject.

Illustrative embodiment 21. The method of any of illustrative embodiments 9-20, further defined as a method of reducing a volume of a tumor and/or preventing an increase of volume of the tumor, wherein the tumor is present in a body of a living subject and includes a plurality of cancer cells, and wherein the at least one cancer cell is isolated from the tumor prior to step (2).

Illustrative embodiment 22. The method of illustrative embodiment 21, further comprising the step of applying an alternating electric field to a target region of the subject prior to isolating the at least one cancer cell, and wherein the target region includes the tumor.

Illustrative embodiment 23. An immunogenic composition, comprising: a population of isolated, antigen-loaded dendritic cells, wherein the antigen-loaded dendritic cells are produced by co-culturing dendritic cells with at least one cancer cell isolated from a subject to produce the antigen-loaded dendritic cells, and wherein the at least one cancer cell has been exposed to an alternating electric field in vivo or ex vivo prior to co-culture with the dendritic cells.

Illustrative embodiment 24. A method of preparing an immunogenic composition, the method comprising the steps of: co-culturing dendritic cells with at least one cancer cell isolated from a subject to produce antigen-loaded dendritic cells, wherein the at least one cancer cell has been exposed to an alternating electric field in vivo or ex vivo prior to co-culture with the dendritic cells; and isolating a population of antigen-loaded dendritic cells to form the immunogenic composition.

Illustrative embodiment 25. A method of preparing an immunogenic composition, the method comprising the steps of: (1) applying an alternating electric field to a target region of the subject; (2) isolating cancer cells from the target region to which the alternating electric field has been applied; (3) co-culturing the isolated cancer cells with dendritic cells to produce antigen-loaded dendritic cells; and (4) isolating antigen-loaded dendritic cells from the co-culture of (3) and from the cancer cells to form the immunogenic composition.

Illustrative embodiment 26. A method of treating cancer in a subject, the method comprising the steps of: (1) applying an alternating electric field to a target region of the subject; (2) isolating cancer cells from the target region to which the alternating electric field has been applied; (3) co-culturing the isolated cancer cells with dendritic cells to produce antigen-loaded dendritic cells; (4) isolating antigen-loaded dendritic cells from the co-culture of (3) and from the cancer cells; and (5) administering the antigen-loaded dendritic cells to the subject.

Illustrative embodiment 27. The method of illustrative embodiment 25 or 26, further comprising the step of isolating the dendritic cells utilized in step (3) or precursors thereof from the subject prior to step (1).

Illustrative embodiment 28. The method of illustrative embodiment 27, wherein isolation of the dendritic cells is further defined as comprising the steps of: isolating immature monocytes (dendritic cell precursors) from blood stream of the subject; generating immature dendritic cells from the immature monocytes/dendritic cell precursors; and culturing the dendritic cell precursors to induce differentiation into mature dendritic cells.

Illustrative embodiment 29. The method of any of illustrative embodiments 25-28, further comprising the step of isolating dendritic cells or precursors thereof from subject following step (1).

Illustrative embodiment 30. The method of any of illustrative embodiments 25-29, wherein the dendritic cells are isolated from an HLA-matched donor.

Illustrative embodiment 31. The method of any of illustrative embodiments 25-30, wherein step (3) is performed in the presence of at least one composition selected from the group consisting of a cytokine, an interferon, granulocyte-macrophage colony-stimulating factor (GM-CSF), CD40 ligand (CD40L), a Toll-like receptor (TLR) agonist, and combinations thereof.

Illustrative embodiment 32. The method of any of illustrative embodiments 26-31, further comprising the step of: (6) applying the alternating electric field to the target region of the subject.

Illustrative embodiment 33. The method of any of illustrative embodiments 26-32, further defined as a method of reducing a volume of a tumor and/or preventing an increase of volume of the tumor, wherein the tumor is present in a body of a living subject and includes a plurality of cancer cells, and wherein step (1) is further defined as applying an alternating electric field to a target region of the subject, wherein the target region includes the tumor.

Illustrative embodiment 34. A method of preparing an immunogenic composition, the method comprising the steps of: (1) isolating at least one cancer cell from the subject; (2) applying an alternating electric field ex vivo to the isolated at least one cancer cell; (3) co-culturing the isolated at least one cancer cell to which the alternating electric field has been applied with dendritic cells to produce antigen-loaded dendritic cells; and (4) isolating antigen-loaded dendritic cells from the co-culture of (3) and from the at least one cancer cell to form the immunogenic composition.

Illustrative embodiment 35. A method of treating cancer in a subject, the method comprising the steps of: (1) isolating at least one cancer cell from the subject; (2) applying an alternating electric field ex vivo to the isolated at least one cancer cell; (3) co-culturing the isolated at least one cancer cell to which the alternating electric field has been applied with dendritic cells to produce antigen-loaded dendritic cells; (4) isolating antigen-loaded dendritic cells from the co-culture of (3) and from the at least one cancer cell; and (5) administering the antigen-loaded dendritic cells to the subject.

Illustrative embodiment 36. The method of illustrative embodiment 34 or 35, wherein the at least one cancer cell is further defined as at least a portion of a tumor.

Illustrative embodiment 37. The method of any of illustrative embodiments 34-36, wherein the dendritic cells are isolated from the subject and/or from an HLA-matched donor.

Illustrative embodiment 38. The method of any of illustrative embodiments 34-37, wherein step (3) is performed in the presence of at least one composition selected from the group consisting of a cytokine, an interferon, granulocyte-macrophage colony-stimulating factor (GM-CSF), CD40 ligand (CD40L), a Toll-like receptor (TLR) agonist, and combinations thereof.

Illustrative embodiment 39. The method of any of illustrative embodiments 35-38, further comprising the step of: (6) applying the alternating electric field to the target region of the subject.

Illustrative embodiment 40. The method of any of illustrative embodiments 35-39, further defined as a method of reducing a volume of a tumor and/or preventing an increase of volume of the tumor, wherein the tumor is present in a body of a living subject and includes a plurality of cancer cells, and wherein steps (1)-(3) are further defined as: (1) resecting at least a portion of the tumor from the subject; (2) applying an alternating electric field ex vivo to at least a portion of the resected tumor; (3) co-culturing the at least a portion of the resected tumor to which the alternating electric field has been applied with dendritic cells to produce antigen-loaded dendritic cells.

Illustrative embodiment 41. The method of any of illustrative embodiments 6-40, wherein the at least one cancer cell is selected from the group consisting of hepatocellular carcinoma cells, glioblastoma cells, pleural mesothelioma cells, differentiated thyroid cancer cells, advanced renal cell carcinoma cells, ovarian cancer cells, pancreatic cancer cells, lung cancer cells, cervical cancer cells, breast cancer cells, and combinations thereof.

Illustrative embodiment 42. The method of any of illustrative embodiments 9-22, 26-33, and 35-41, wherein the antigen-loaded dendritic cells are administered intradermally, subcutaneously, intravenously, and/or intranodally.

Illustrative embodiment 43. The method of any of illustrative embodiments 9-22, 26-33, and 35-42, wherein the antigen-loaded dendritic cells are administered to the subject in the form of at least one immunogenic composition, and wherein the at least one immunogenic composition further comprises at least one compound selected from the group consisting of an adjuvant, a cytokine, an interferon, a TLR agonist, a STING (stimulator of interferon genes) agonist, GM-CSF, CD40L, Fms related tyrosine kinase 3 ligand (FLT3L), a C type Lectin Receptor (CLR), an anti-LAG3 agent (such as, but not limited to, OPDUALAG™ and/or Relatimab (Bristol-Myers Squibb, New York, NY)), and combinations thereof.

Illustrative embodiment 44. An immunogenic composition, comprising: a population of isolated, antigen-loaded dendritic cells produced by the method of any of illustrative embodiments 3-8, 10-19, 24-25, 27-31, 34, 36-38, and 41.

Illustrative embodiment 45. The method or immunogenic composition of any of illustrative embodiments 1-44, wherein at least one of: the alternating electric field is applied at a frequency in a range of from about 50 kHz to about 1 MHz; the alternating electric field has a field strength of at least about 1 V/cm in at least a portion of the cancer cells; and the period of time that the alternating electric field is applied is at least about 24 hours.

Illustrative embodiment 46. The method or immunogenic composition of illustrative embodiment 45, wherein the alternating electric field is applied at a frequency in a range of from about 50 kHz to about 500 kHz, or a range of from about 50 kHz to about 190 kHz, or a range of from about 50 kHz to about 180 kHz, or a range of from about 50 kHz to about 175 kHz, or a range of from about 50 kHz to about 160 kHz, or a range of from about 50 kHz to about 150 kHz.

Illustrative embodiment 47. The method or immunogenic composition of illustrative embodiment 46, wherein the alternating electric field is applied at a frequency of about 150 kHz and a field strength of about 2.7 V/cm for a period of about 48 hours.

Illustrative embodiment 48. The immunogenic composition of any of illustrative embodiments 23 and 44-47, further comprising a pharmaceutically acceptable carrier.

Illustrative embodiment 49. The immunogenic composition of any of illustrative embodiments 23 and 44-48, wherein the immunogenic composition is formulated for intradermal, subcutaneous, intravenous, and/or intranodal administration.

Illustrative embodiment 50. The immunogenic composition of any of illustrative embodiments 23 and 44-49, further comprising at least one composition selected from the group consisting of an adjuvant, a cytokine, an interferon, a TLR agonist, a STING (stimulator of interferon genes) agonist, GM-CSF, CD40L, Fms related tyrosine kinase 3 ligand (FLT3L), a C type Lectin Receptor (CLR), an anti-LAG3 agent, and combinations thereof.

Illustrative embodiment 51. The method or immunogenic composition of any of illustrative embodiments 1-50, wherein the dendritic cells comprise at least one of conventional DC1 (cDC1), cDC2, and plasmacytoid DC (pDC).

Illustrative embodiment 52. Use of the immunogenic composition of any of illustrative embodiments 23 and 44-51 in a method of treating cancer.

Illustrative embodiment 53. Use of an immunogenic composition in a method of treating cancer, wherein the use comprises the method of any of illustrative embodiments 9-22, 26-33, 35-43, 45-47, and 51.

While the above disclosures describe the inventive concept(s) in conjunction with the specific experimentation, results, and language set forth, it is evident that many alternatives, modifications, and variations will be apparent to those skilled in the art. Accordingly, it is intended to embrace all such alternatives, modifications, and variations that fall within the spirit and broad scope of the present disclosure.

1-19. (canceled)

20. A method of preparing an immunogenic composition, the method comprising the steps of:
(1) applying an alternating electric field to a target region of the subject;
(2) isolating cancer cells from the target region to which the alternating electric field has been applied;
(3) co-culturing the isolated cancer cells with dendritic cells to produce antigen-loaded dendritic cells; and
(4) isolating antigen-loaded dendritic cells from the co-culture of (3) and from the cancer cells to form the immunogenic composition.

21. (canceled)

22. The method of claim 20, further comprising the step of isolating the dendritic cells utilized in step (3) or precursors thereof from the subject prior to step (1).

23. The method of claim 20, wherein the dendritic cells are isolated from an HLA-matched donor.

24. The method of claim 20, wherein step (3) is performed in the presence of at least one composition selected from the group consisting of a cytokine, an interferon, granulocyte-macrophage colony-stimulating factor (GM-CSF), CD40 ligand (CD40L), a Toll-like receptor (TLR) agonist, and combinations thereof.

25. The method of claim 20, further comprising the step of:
(6) applying the alternating electric field to the target region of the subject.

26-31. (canceled)

32. The method of claim 20, wherein the at last cancer cells are selected from the group consisting of hepatocellular carcinoma cells, glioblastoma cells, pleural mesothelioma cells, differentiated thyroid cancer cells, advanced renal cell carcinoma cells, ovarian cancer cells, pancreatic cancer cells, lung cancer cells, cervical cancer cells, breast cancer cells, and combinations thereof.

33. (canceled)

34. The method of claim 21, wherein the antigen-loaded dendritic cells are administered to the subject in the form of at least one immunogenic composition, and wherein the at least one immunogenic composition further comprises at least one compound selected from the group consisting of an adjuvant, a cytokine, an interferon, a TLR agonist, a STING (stimulator of interferon genes) agonist, GM-CSF, CD40L, Fms related tyrosine kinase 3 ligand (FLT3L), a C type Lectin Receptor (CLR), an anti-LAG3 agent (such as, but not limited to, OPDUALAG™ and/or Relatimab (Bristol-Myers Squibb, New York, NY)), and combinations thereof.

35. (canceled)

36. The method of claim 1, wherein the alternating electric field is applied at a frequency in a range of from about 50 kHz to about 1 MHz.

37-41. (canceled)

42. The method of claim 1, wherein the dendritic cells comprise at least one of conventional DC 1 (cDC1), cDC2, and plasmacytoid DC (pDC).

43-44. (canceled)

45. An immunogenic composition, comprising:
a population of isolated, antigen-loaded dendritic cells produced by the method of claim 1.

46. The immunogenic composition of claims 45, further comprising at least one composition selected from the group consisting of an adjuvant, a cytokine, an interferon, a TLR agonist, a STING (stimulator of interferon genes) agonist, GM-CSF, CD40L, Fms related tyrosine kinase 3 ligand (FLT3L), a C type Lectin Receptor (CLR), an anti-LAG3 agent, and combinations thereof.

47. The method or immunogenic composition of claim 45, wherein the dendritic cells comprise at least one of conventional DC 1 (cDC1), cDC2, and plasmacytoid DC (pDC).

48. A method of treating cancer in a subject, the method comprising the steps of:
(1) applying an alternating electric field to a target region of the subject;
(2) isolating cancer cells from the target region to which the alternating electric field has been applied;
(3) co-culturing the isolated cancer cells with dendritic cells to produce antigen-loaded dendritic cells;
(4) isolating antigen-loaded dendritic cells from the co-culture of (3) and from the cancer cells; and
(5) administering the antigen-loaded dendritic cells to the subject.

49. The method of claim 48, further comprising the step of isolating the dendritic cells utilized in step (3) or precursors thereof from the subject prior to step (1).

50. The method of claim 48, wherein the dendritic cells are isolated from an HLA-matched donor.

51. The method of claim 48, wherein the dendritic cells comprise at least one of conventional DC 1 (cDC1), cDC2, and plasmacytoid DC (pDC).

51. The method of claim 48, wherein step (3) is performed in the presence of at least one composition selected from the group consisting of a cytokine, an interferon, granulocyte-macrophage colony-stimulating factor (GM-CSF), CD40 ligand (CD40L), a Toll-like receptor (TLR) agonist, and combinations thereof.

52. The method of claim 48, further comprising the step of:
(6) applying the alternating electric field to the target region of the subject.

53. The method of claim 48, wherein the cancer cells are selected from the group consisting of hepatocellular carcinoma cells, glioblastoma cells, pleural mesothelioma cells, differentiated thyroid cancer cells, advanced renal cell carcinoma cells, ovarian cancer cells, pancreatic cancer cells, lung cancer cells, cervical cancer cells, breast cancer cells, and combinations thereof.

54. The method of claim 48, wherein the antigen-loaded dendritic cells are administered to the subject in the form of at least one immunogenic composition, and wherein the at least one immunogenic composition further comprises at least one compound selected from the group consisting of an adjuvant, a cytokine, an interferon, a TLR agonist, a STING (stimulator of interferon genes) agonist, GM-CSF, CD40L, Fms related tyrosine kinase 3 ligand (FLT3L), a C type Lectin Receptor (CLR), an anti-LAG3 agent (such as, but not limited to, OPDUALAG™ and/or Relatimab (Bristol-Myers Squibb, New York, NY)), and combinations thereof.

55. The method of claim 48, wherein the alternating electric field is applied at a frequency in a range of from about 50 kHz to about 1 MHz.
